(19) United States
(12) Patent Application Publication
(10) Pub. No.: US 2012/0318679 A1
(43) Pub. Date: Dec. 20, 2012

(54) METHODS AND COMPOSITIONS COMPRISING MACROCYCLES, INCLUDING HALOGENATED MACROCYCLES

(75) Inventors: Daniel G. Nocera, Winchester, MA (US); Dilek Dogutan Kiper, Cambridge, MA (US); Robert McGuire, JR., Brighton, MA (US); Changhoon Lee, Palo Alto, CA (US)

(73) Assignee: Massachusetts Institute of Technology, Cambridge, MA (US)

(21) Appl. No.: 13/474,623

(22) Filed: May 17, 2012

Related U.S. Application Data
(60) Provisional application No. 61/487,217, filed on May 17, 2011.

(51) Int. Cl. C07F 15/06; C25B 3/12; B01J 19/12

(52) U.S. Cl. 205/423; 540/145; 204/157.6; 205/457

(57) ABSTRACT

The present invention relates generally to methods and compositions comprising macrocycles. In some cases, at least one beta-position of the macrocycle comprises an electron-withdrawing group, for example, a halide. In some embodiments, methods for forming and/or modifying a macrocycle using microwave energy are provided. In some embodiments, the compositions are employed in catalysis reactions.

### RELATED APPLICATIONS

This application claims the benefit of U.S. Provisional Patent Application Ser. No. 61/487,217, filed May 17, 2011, entitled “METHODS AND COMPOSITIONS COMPRISING MACROCYCLES, INCLUDING HALOGENATED MACROCYCLES,” by Nocera, et al., incorporated herein by reference.

### FIELD OF THE INVENTION

The present invention relates generally to methods and compositions comprising macrocycles. In some embodiments, at least one beta-position of the macrocycle comprises an electron-withdrawing group, for example, a halide.

### BACKGROUND OF THE INVENTION

The activation of many small molecules requires the coupling of electrons to protons. In the absence of such coupling, large reaction barriers confront the conversion of the small molecule. The challenge to effecting proton-coupled electron transfer (PCET) is in the management of the disparate tunneling length scales of the electron and proton. Proton transfer is fundamentally limited to short distances whereas the lighter electron may transfer over much longer distances. Hangman porphyrins (or other macrocycles) can manage the proton and electron by establishing the proton transfer distance with an acid-base group poised above the electron transfer conduit of the porphyrin macrocycle. Electron or energy transfer to the macrocycle can be coupled to a short proton transfer to or from substrates bound within the hangman cleft.

While methods exist for the synthesis of porphyrins or other macrocycles comprising various pendent groups (e.g., comprising dibenzofuran, xanthene), the methods generally are low yielding and/or do not allow for the synthesis of a wide range of substituted macrocycles. In addition, macrocycles have not been employed in a wide range of applications, for example, catalysis.

Accordingly, improved methods, compositions, and systems are needed.

### SUMMARY OF THE INVENTION

In some embodiments, a composition is provided having the formula:

X-Y;

wherein X comprises a macrocycle having 2-7 heteroatoms positioned such that at least some of the heteroatoms are able to coordinate a metal or a semi-metal within a central binding cavity of the macrocycle; and Y is a pendent group, optionally substituted, wherein at least one beta-position of the macrocycle is an electron-withdrawing group.

In some embodiments, a method is provided comprising forming a mixture of a metal complex comprising a metal atom and a composition having the formula:

X-Y;

wherein X comprising a macrocycle having 2-7 heteroatoms positioned such that at least some of the heteroatoms are able to coordinate a metal within a central binding cavity of the macrocycle, wherein at least one beta-position of the macrocycle is an electron-withdrawing group, optionally substituted; and Y is a pendent group; and exposing said mixture to microwave energy, thereby forming a compound comprising the macrocycle and the metal atom, wherein the metal atom is coordinated by at least some of the heteroatoms within the central binding cavity of the macrocycle.

In some embodiments, a method of catalysis is provided comprising providing a composition having the formula:

X-Y;

wherein X comprises a macrocycle having 2-7 heteroatoms positioned such that at least some of the heteroatoms are able to coordinate a metal within a central binding cavity of the macrocycle, wherein at least one beta-position of the macrocycle is an electron-withdrawing group; and Y is a pendent group, optionally substituted; wherein at least one metal atom is coordinated by the macrocycle and/or the pendent group; and exposing the composition to a reactant, wherein a product is formed from the reactant following application of a voltage to the composition.

In some embodiments, a method of forming oxygen gas from water is provided comprising providing a composition, having the formula:

X-Y;

wherein X comprises a macrocycle having 2-7 heteroatoms positioned such that at least some of the heteroatoms are able to coordinate a metal within a central binding cavity of the macrocycle, wherein at least one beta-position of the macrocycle is an electron-withdrawing group; and Y is a pendent group; wherein at least one metal atom is coordinated by the macrocycle and/or the pendent group; and exposing the composition to water, wherein oxygen gas is formed from water following application of a voltage to the composition.

In some embodiments, a method of forming hydrogen gas from water is provided comprising providing a composition, having the formula:

X-Y;

wherein X comprises a macrocycle having 2-7 heteroatoms positioned such that at least some of the heteroatoms are able to coordinate a metal within a central binding cavity of the macrocycle; and Y is a pendent group; wherein at least one metal atom is coordinated by the macrocycle and/or the pendent group; and exposing the composition to water, acid, organic solvent, or combination thereof, wherein hydrogen gas is formed from the water, acid, organic solvent, or combination thereof following application of a voltage to the composition. In some cases, at least one beta-position of the macrocycle is an electron-withdrawing group.

In some embodiments, a method of reducing CO₂ is provided comprising providing a composition, having the formula:

X-Y;

wherein X comprises a macrocycle having 2-7 heteroatoms positioned such that at least some of the heteroatoms are able to coordinate a metal within a central binding cavity of the macrocycle, wherein at least one beta-position of the macrocycle is an electron-withdrawing group; Y is a pendent group; wherein at least one metal atom is coordinated by the macrocycle and/or the pendent group; exposing the composition to CO₂, wherein the CO₂ is reduced following application of a voltage to the composition.

In some embodiments, Y is substituted with at least one —Z—P_{g}, wherein Z is a hydrolyzable group, and P_{g} is a protecting group; and following exposure to microwave energy, Y is substituted with -Z-D_{g}, wherein D is a deprotected group or optionally absent. In some embodiments, the method further comprising reacting the compound following exposure to microwave energy having comprising the formula -Y-Z-D_{g}, to form a compound having the formula —Y—Z—H. In some embodiments, Y is substituted with G, and wherein following exposing said mixture to microwave energy to form a compound comprising the macrocycle and the metal atom, wherein the metal atom is coordinated by at least some of the heteroatoms within the central binding cavity of the macrocycle and G.

In some embodiments, the electron-withdrawing group is selected from the group consisting of halide, NO₂, and CN. In some embodiments, the electron-withdrawing group is halide. In some embodiments, the electron-withdrawing group is selected from the group consisting of halide, NO₂ and CN. In some embodiments, the electron-withdrawing group is halide.

In some embodiments, Y is substituted with at least one —Z—P_{g}, wherein Z is a hydrolyzable group, and P_{g} is a protecting group; and following exposure to microwave energy, Y is substituted with -Z-D_{g}, wherein D_{g} is a deprotected group or optionally absent. In some embodiments, the method further comprising reacting the compound following exposure to microwave energy having comprising the formula -Y-Z-D_{g}, to form a compound having the formula —Y—Z—H. In some embodiments, wherein Y is substituted with G, and wherein following exposing said mixture to microwave energy to form a compound comprising the macrocycle and the metal atom, wherein the metal atom is coordinated by at least some of the heteroatoms within the central binding cavity of the macrocycle and G.

In some embodiments, the macrocycle comprises a compound having the structure:

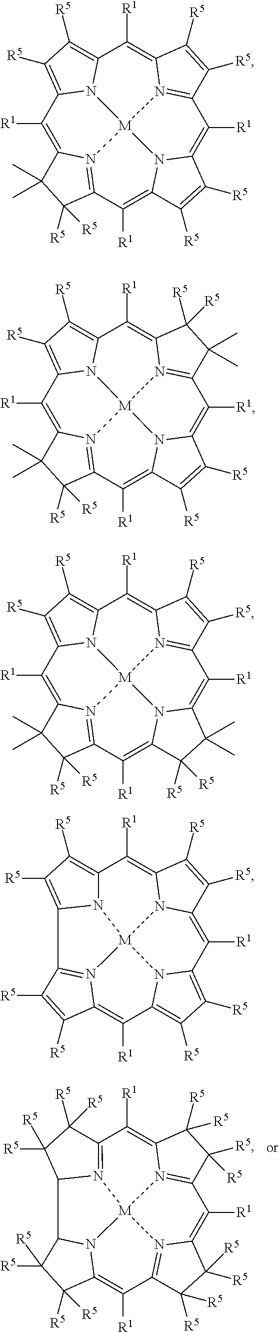

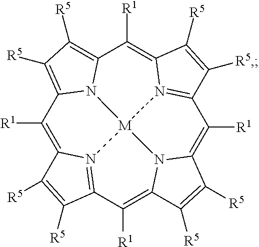

wherein each R¹ can be the same or different and is selected from the group consisting of hydrogen, alkyl, aryl, heteroalkyl, heteroaryl, alkenyl, alkynyl, cycloalkyl, cycloalkylalkyl, cycloalkylalkenyl, cycloalkylalkynyl, acyl, carboxylic acid, acylamino, alkylthio, amino, alkylamino, arylalkylamino, alkoxy, arylalkyl, or alkylaryl, each optionally substituted provided at least one R¹ is a group comprising of the formula -Y; wherein each R⁵ can be the same or different and is hydrogen, halide, CN, CO₂, alkyl, aryl, heteroalkyl, heteroaryl, alkenyl, alkynyl, cycloalkyl, cycloalkylalkyl, cycloalkylalkenyl, cycloalkylalkynyl, acyl, carboxylic acid, carboxylate, OH, acylamino, alkylthio, amino, alkylamino, arylalkylamino, or alkoxy, each optionally substituted; and M is a metal atom, a semi-metal atom, or at least one hydrogen.

In some embodiments, the macrocycle comprises a compound having the structure:

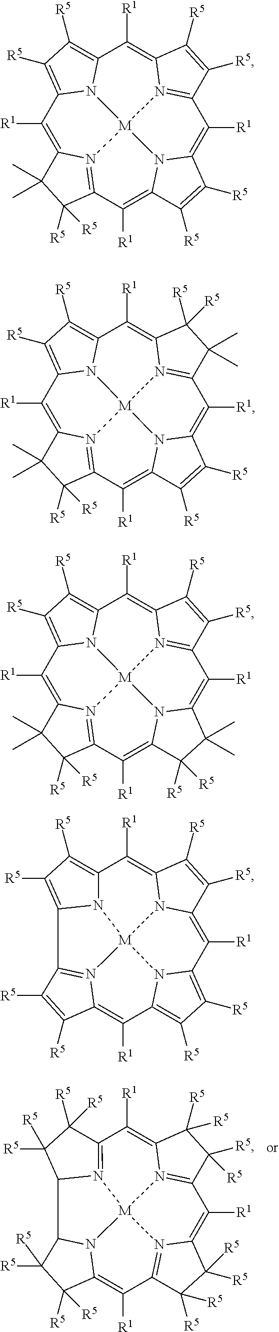

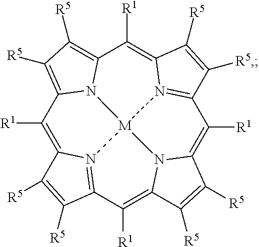

wherein each R¹ can be the same or different and is selected from the group consisting of hydrogen, alkyl, aryl, heteroalkyl, heteroaryl, alkenyl, alkynyl, cycloalkyl, cycloalkylalkyl, cycloalkylalkenyl, cycloalkylalkynyl, acyl, carboxylic acid, acylamino, alkylthio, amino, alkylamino, arylalkylamino, alkoxy, arylalkyl, or alkylaryl, each optionally substituted provided at least one R¹ is a group comprising of the formula -Y; wherein each R⁵ can be the same or different and is hydrogen, halide, CN, CO₂, alkyl, aryl, heteroalkyl, heteroaryl, alkenyl, alkynyl, cycloalkyl, cycloalkylalkyl, cycloalkylalkenyl, cycloalkylalkynyl, acyl, carboxylic acid, carboxylate, OH, acylamino, alkylthio, amino, alkylamino, arylalkylamino, or alkoxy, each optionally substituted, provided at least one R⁵ is an electron-withdrawing group; and M is a metal atom, a semi-metal atom, or at least one hydrogen.

In some embodiments, the at least one Y is substituted by -G, or —Z—P_{g}, or -Z-D_{g}, or —Z—H. In some embodiments, the at least one Y is substituted by —COOR², wherein R² is hydrogen, alkyl, aryl, heteroalkyl, heteroaryl, or a protecting group, each optionally substituted. In some embodiments, each R⁵ is an electron-withdrawing group. In some embodiments, each R⁵ is a halide. In some embodiments, each R⁵ is a fluoride.

In some embodiments, the macrocycle is selected from the group consisting of a porphycene, a [18]porphyrin(2.1.0.1), an N-confused porphyrin, a sapphyrin, a heterosapphyrin, a rubyrin, an orangarin, a cycle[8]pyrrole, a rosarin, a turcasarin, a texaphyrin, a cryptan, a calixphyrin, or a catenane, each optionally substituted.

In some embodiments, —Z—P_{g} is selected from the group consisting of —COOP_{g}, —PO(OR)(OP_{g}), —B(OR)(OP_{g}), —CO(NR)(NP_{g}), —NRP_{g}, —C(NR₂)(NRP_{g}), and —OP_{g}, wherein R is a suitable organic substituent and P_{g} is a protecting group. In some embodiments, -Z-D_{g} is selected from the group consisting of —COOD_{g}, —PO(OR)(OD_{g}), —B(OR)(OD_{g}), —CO(NR)(ND_{g}), —NRD_{g}, —C(NR₂)(NR)D_{g}), —OD_{g}, wherein R is a suitable organic substituent and D_{g} is a deprotected group or optionally absent. In some embodiments, -G is a substituent which is capable of coordinating a metal atom or semi-metal atom, but is not hydrolysable. In some embodiments, -G is a substituent comprising a lone pair of electrons which is capable of interacting with a metal or a semi-metal coordinated within the central binding cavity of the macrocycle. In some embodiments, -G is selected from the group consisting of amidine, alcohol, amine, amide, heteroaryl, sulfone, amidine, or sulfide. In some embodiments, deprotected group (D_{g}) is a cation. In some embodiments, the deprotected group (D_{g}) is K⁺ or Na⁺.

In some embodiments, M is a metal atom. In some embodiments, the metal atom is selected from the group consisting of chromium, manganese, titanium, vanadium, iron, cobalt, nickel, copper, or zinc. In some embodiments, the metal atom is selected from the second or third row transition metals. In some embodiments, the metal atom is a lanthanide. In some embodiments, M is a semi-metal atom. In some embodiments, the semi-metal atom is selected from the group consisting of boron, silicon, germanium, arsenic, antimony, or tellurium. In some embodiments, the metal atom is cobalt, zinc, or iron. In some embodiments, the metal atom is coordinated by at least some of the heteroatoms is further coordinated with at least on auxiliary ligand. In some embodiments, the auxiliary ligand is a halide or a coordinating solvent.

In some embodiments, Y comprises xanthene, dibenzofuran, biphenylene, or anthracene. In some embodiments, Y has the structure:

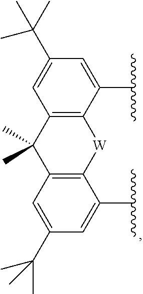

wherein W is a heteroatom. In some embodiments, W is O. In some embodiments, Y is selected from the group consisting of alkyl, heteroaryl, cycloalkyl, heterocyclolkyl, aryl, alkylaryl, arylalkyl, alkoxy, amino, heteroalkyl, each optionally substituted. In some embodiments, Y comprises a plurality of fused aryl, heteroaryl, cycloalkyl, and/or heterocycloalkyl rings, each optionally substituted.

In some embodiments, the composition comprises at least one substituent which aids in increasing the water solubility of the composition.

In some embodiments, the composition or mixture is exposed to microwave energy for about 1 minute, about 2 minutes, about 3 minutes, about 5 minutes, about 10 minutes, about 15 minutes, about 20 minutes, about 30 minutes, about 1 hour, about 2 hours, about 4 hours, about 8 hours, about 12 hours, about 18 hours, or about 24 hours. In some embodiments, the composition or mixture is exposed to microwave energy at a temperature of about 60° C., about 65° C., about 70° C., about 75° C., about 80° C., about 65° C., about 90° C. In some embodiments, the composition or mixture is exposed to microwave energy at a temperature of between about 20° C. and about 100° C., between about 30° C. and about 90° C., between about 40° C. and about 80° C., between about 60° C. and about 90° C., between about 65° C. and about 85° C., between about 70° C. and about 85° C., or between about 65° C. and about 75° C. In some embodiments, the composition or mixture is provided as a solution comprising acetonitrile, tetrahydrofuran, chloroform, dichloromethane, toluene, methanol, dimethylformamide, or combinations thereof. In some embodiments, the microwave energy is applied at a transmit power level of no more than about 5 W, about 10 W, about 15 W, about 20 W, about 50 W, about 100 W, about 200 W, about 400 W, about 500 W, about 750 W, or about 1000 W. In some embodiments, the microwave energy is applied at a power density of no more than about 5 W/m², about 10 W/m², about 15 W/m², about 20 W/m², about 50 W/m², about 100 W/m², about 200 W/m², about 400 W/m², about 500 W/m², about 750 W/m², or about 1000 W/m².

In some embodiments, -Y-Z-D_{g} is further reacted with an acid to form a compound having the formula —Y—Z—H. In some embodiments, -Y-Z-D has the formula —Y—COOD_{g} and —Y—Z—H has the formula —Y—COOH. In some embodiments, the acid is HCl. In some embodiments, the acid is provided as an aqueous solution. In some embodiments, the composition is provided as a solution. In some embodiments, the solution further comprises a base. In some embodiments, the base is KOH or NaOH. In some embodiments, the base is present in a concentration of between about 2 N and about 10 N, or between about 4 N and about 8 N, or at about 6 N.

In some embodiments, the macrocycle comprises more than one pendant group. In some embodiments, G comprises a crown molecule. In some embodiments, the crown molecule is associated with a cation. In some embodiments, the composition comprises at least one water-solubilizing group. In some embodiments, at least one R¹ or R⁵ comprises a water-solubilizing group. In some embodiments, the water-solubilizing group is an ionic group.

### BRIEF DESCRIPTION OF THE DRAWINGS

Non-limiting embodiments of the present invention will be described by way of example with reference to the accompanying figures, which are schematic and are not intended to be drawn to scale. Unless indicated as representing the prior art, the figures represent aspects of the invention. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1 and 2 show non-limiting examples of macrocycles.

FIGS. 3a, 4, 5, 7a, 8a, 9a, and 9c show cyclic voltammograms (CVs) of non-limiting compounds, according to some embodiments.

FIG. 3b shows mass spectrometric detection of O₂ and CO₂ during electrolysis, according to a non-limiting embodiment.

FIG. 6 shows a schematic of hydrogen production using a non-limiting compound, according to one embodiment.

FIG. 7b shows a plot of the difference between anodic or cathodic peak potential and midpoint potential (Eₚ−E^{o}) vs. log of scan rate, according to a non-limiting embodiment.

FIG. 8b shows a plot of (Eₚ−E^{o})F/RT vs. In of scan rate, according to a non-limiting embodiment.

FIGS. 9b and 9d shows working curves generated by simulating CVs, according to some embodiments.

### DETAILED DESCRIPTION

The present invention generally relates to methods and composition comprising macrocycles. In some embodiments, at least one beta-position of the macrocycle comprises an electron-withdrawing group, for example, a halide. The subject matter of the present invention involves, in some cases, interrelated products, alternative solutions to a particular problem, and/or a plurality of different uses of one or more systems and/or articles.

In some embodiments, compositions comprising macrocycles are provided. In some cases, a macrocycle comprises at least one electron-withdrawing group, for example, a halide. For example, in some embodiments, a composition comprises the formula:

X-Y;

wherein X comprises a macrocycle and Y is a pendent group, optionally substituted, wherein at least one beta-position of the macrocycle comprises or is an electron-withdrawing group, for example, a halide.

The term “macrocycle” (e.g., “X”), as used herein, is given its ordinary meaning in the art and generally refers to a macrocyclic molecule of repeating units of carbon atoms and heteroatoms (e.g., O, S, Se, NR, and/or NH), wherein the heteroatoms are separated by carbon atoms (e.g., generally by at least two and/or three carbon atoms). In some cases, the macrocycle has 2-7 heteroatoms positioned such that at least some of the heteroatoms are able to coordinate a metal atom or semi-metal atom within a central binding cavity of the macrocycle. In some embodiments, the macrocycle comprises between 2 and 7, or between 3 and 6, or 2, or 3, or 4, or 5, or 6, or 7 heteroatoms, wherein the heteroatoms are present in the main ring (e.g., not in a substituent on the ring). In some embodiments, a macrocyclic ring contains at least 8, 9, 10, 11, 12, 13, 14, 15, 16, 17, 18 or more carbon atoms and/or heteroatoms (e.g., O, S, Se, NR, NH), each heteroatom in the ring being separated from adjoining heteroatoms in the ring by two or more carbon atoms. A macrocycle may be optionally substituted and/or may be fused to additional rings (e.g., 1, 2, 3, 4, 5, 6, 7, etc., additional rings such as phenylene, biphenylene, naphthylene, phenanthrylene, and anthrylene rings). In some cases, a macrocycle may comprise a plurality of rings (e.g., comprising a heteroatom) covalently coupled to each other, optionally through linkers (e.g., at least two or three carbon atoms). A non-limiting example of a macrocycle comprising oxygen is a crown ether.

In some cases, a macrocycle may be capable of incorporating a metal atom or semi-metal within a central binding cavity (e.g., core) of the macrocycle. In some cases, the metal atom or semi-metal atom may be charged (e.g., cationic). Additionally, in some instances, at least one auxiliary ligand may be associated with the metal atom or semi-metal atom, as described herein. The at least one auxiliary ligand may be found above and/or below the core (e.g., as apical ligands).

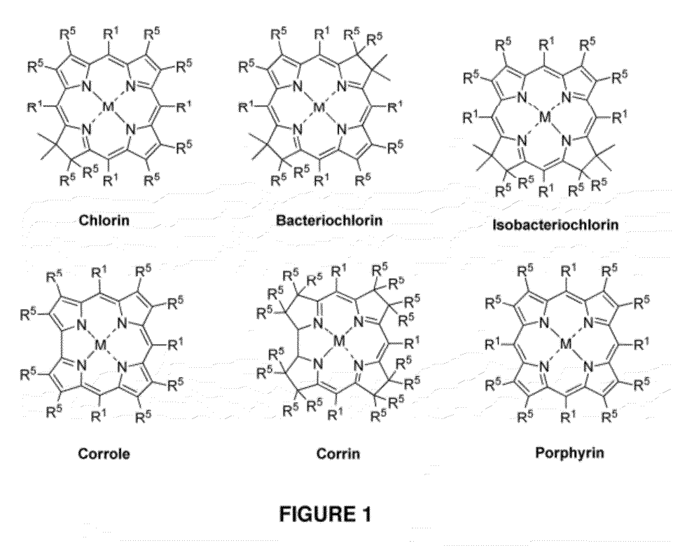

In some embodiments, the heteroatoms comprised in a macrocycle are nitrogen atoms. Non-limiting examples of macrocycles comprising nitrogen atoms include porphyrins, chlorins, bacteriochlorins, isobacteriochlorins, corroles, corrin, phlorin and derivatives, oxophlorin and derivatives, tetraza compounds, porphyrinogen and derivatives, or the like. Non-limiting examples of some nitrogen-containing macrocycles are shown in FIG. 1, wherein each R¹ can be the same or different and is independently selected from the group consisting of hydrogen, alkyl, aryl, heteroalkyl, heteroaryl, alkenyl, alkynyl, cycloalkyl, cycloalkylalkyl, cycloalkylalkenyl, cycloalkylalkynyl, acyl, carboxylic acid, acylamino, alkylthio, amino, alkylamino, arylalkylamino, arylalkyl, alkylaryl, or alkoxy, each optionally substituted, and each M is a metal atom, a semi-metal atom, and/or one or more hydrogen atoms, provided at least one R¹ comprises a pendant group (e.g., Y). In some cases, a macrocycle may comprise more than one pendant group, for example, two, three, four, or more, pendant groups. Pendant groups are described herein.

As will be understood by those of ordinary skill in the art, in some embodiments, wherein not every R¹ is identical, various isomers may form or be present. For example, in the case of a corrole, wherein only three R¹ groups are present, if more than one type of R¹ is present, various isomers can be present/formed. As a specific example, if two of a first type of R¹ group is present and one of a second type of R¹ group is present, a trans isomer (e.g., wherein the two of the first type of R¹ group are on opposite sides of the macrocycle) may be present/formed, and/or a cis isomer (e.g., wherein the two of a first type of R¹ group are on adjacent sides of the macrocycle) may be present/formed. In some cases, the trans isomer is the primary isomer present/formed. In some cases, the trans isomer is exclusively formed/present (e.g., greater than about or about 99.5%, or greater than about or about 99.8%, or greater than about or about 99.9%, or more).

In some embodiments, at least one R¹ comprises has the structure:

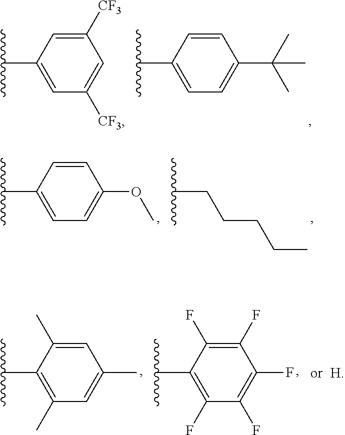

Macrocycles used in connection with the present invention may be further substituted, as will be understood by those of ordinary skill in the art (e.g., for nitrogen containing macrocycles, the beta-pyrrolic positions may be further substituted). For example, as shown in FIG. 1, in some cases, each R⁵ can be the same or different and is hydrogen, halide, NO₂, CN, alkyl, aryl, heteroalkyl, heteroaryl, alkenyl, alkynyl, cycloalkyl, cycloalkylalkyl, cycloalkylalkenyl, cycloalkylalkynyl, acyl, carboxylic acid, carboxylate, OH, acylamino, alkylthio, amino, alkylamino, arylalkylamino, alkoxy, each optionally substituted. In some cases, at least one R⁵ is not hydrogen such that the macrocycle is further substituted.

In some embodiments, at least one beta-position of the macrocycle comprises an electron-withdrawing group. Without wishing to be bound by theory, the present of one or more electron-withdrawing groups on the macrocycle may increase oxidizing power of the macrocycle, which may be useful in embodiments where the macrocycle is being employed for catalysis. The term “electron-withdrawing group” is recognized in the art and as used herein means a functionality which draws electrons to itself more than a hydrogen atom would at the same position. Exemplary electron-withdrawing groups include nitro, cyano, carbonyl groups (e.g., aldehydes, ketones, esters, etc.), sulfonyl, trifluoromethyl, and the like. In some cases, at least one beta-position of the macrocycle is a halide, or CN, or NO₂. In some cases, at least one beta-position of the macrocycle is a halide. For example, with reference to the macrocycles shown in FIG. 1, at least one R⁵ is a halide. In some cases, all or substantially all R⁵ are an electron-withdrawing group. In some cases, at least one R⁵ is an electron-withdrawing group (e.g., halide). In some cases, each R⁵ is independently H, F, Cl, Br, or I, provided at least one R⁵ is F, Cl, Br, or I. In some cases, each R⁵ is independently H or F, provided at least one R⁵ is F. In some cases, each R⁵ is independently F, Cl, Br, or I. In some cases, each R⁵ is F. While much of the discussion herein focuses on electron-withdrawing groups being a halide, this is by no means limiting, and other electron withdrawing groups may be substituted for the halide (e.g., CN, NO₂).

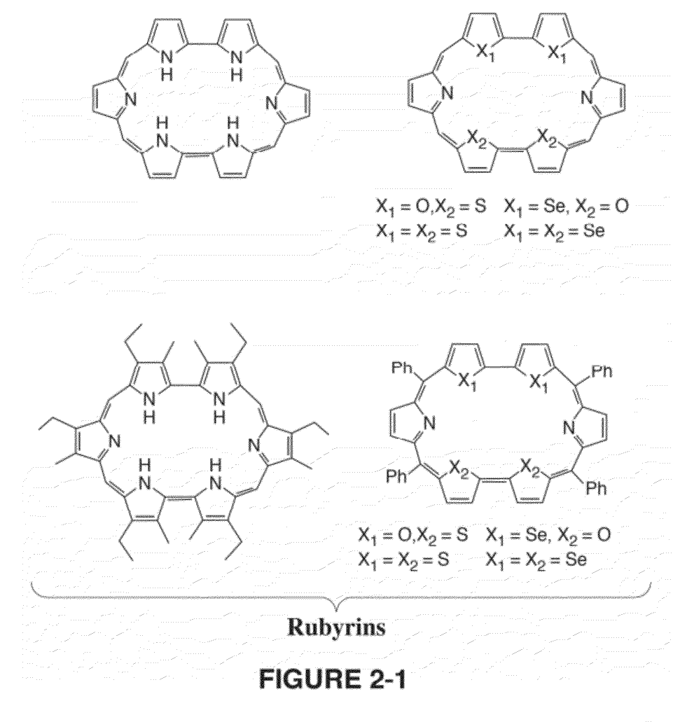

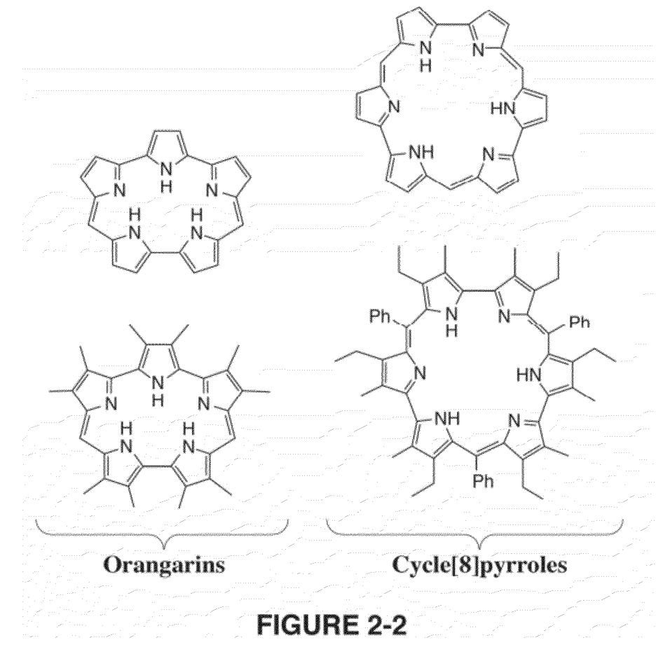

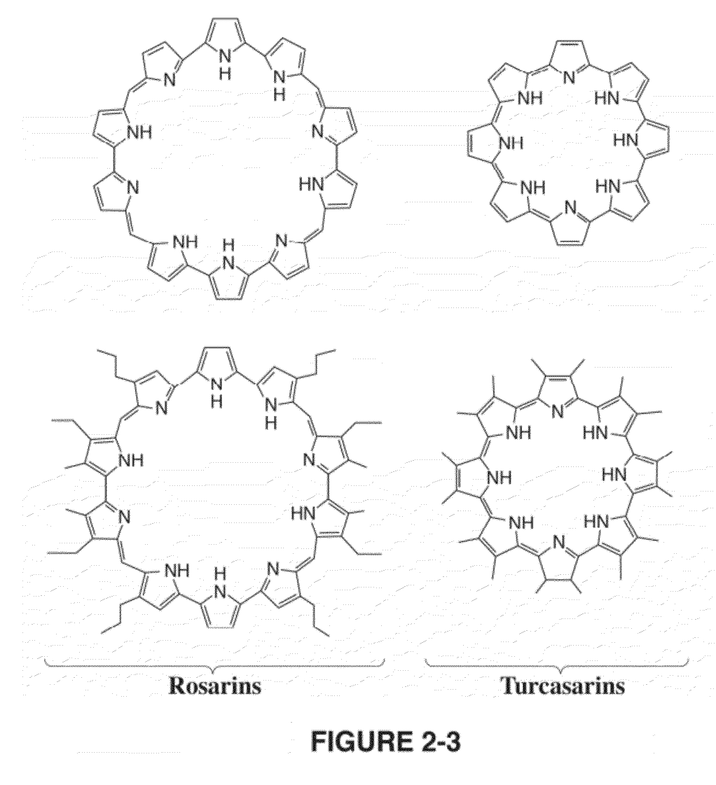

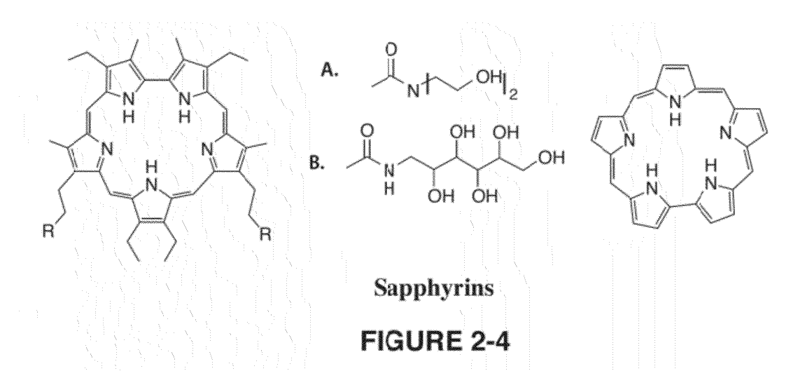

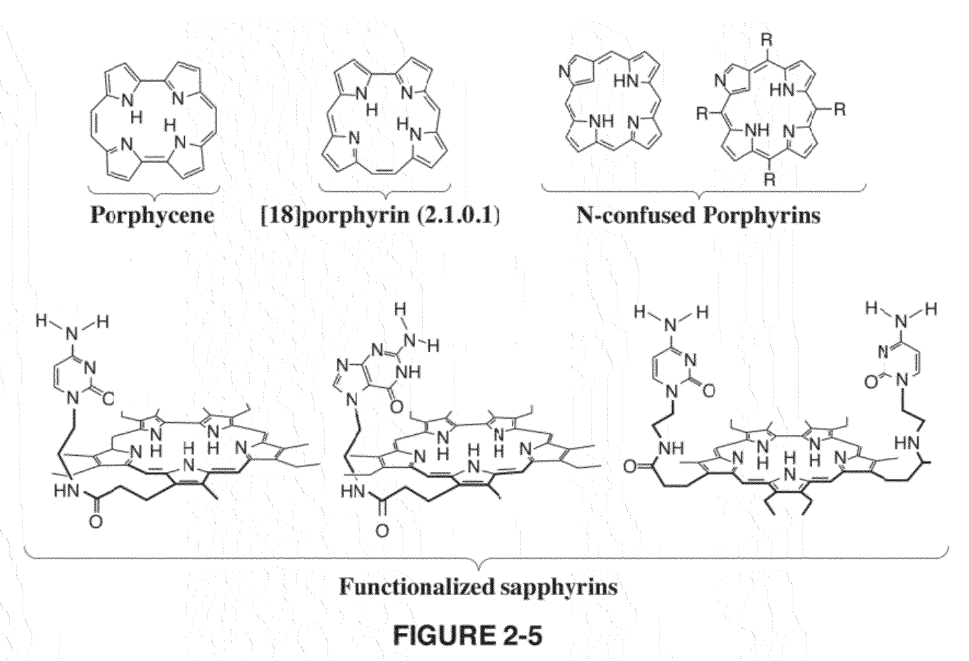

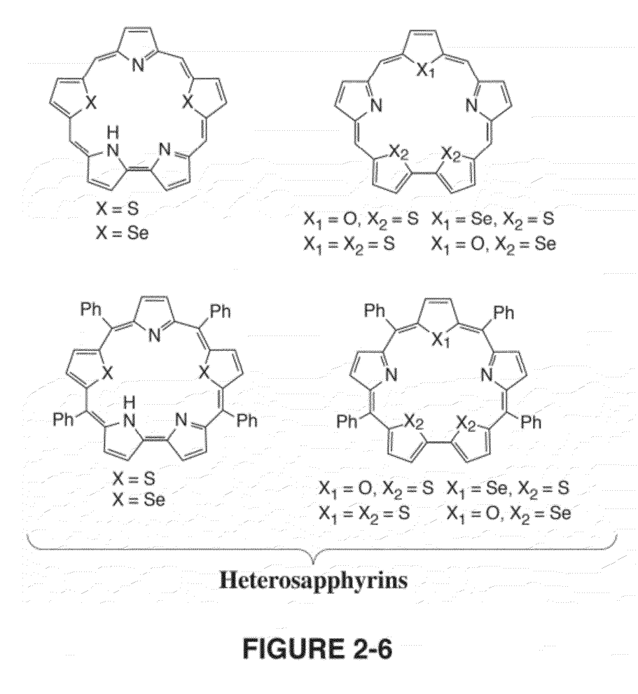

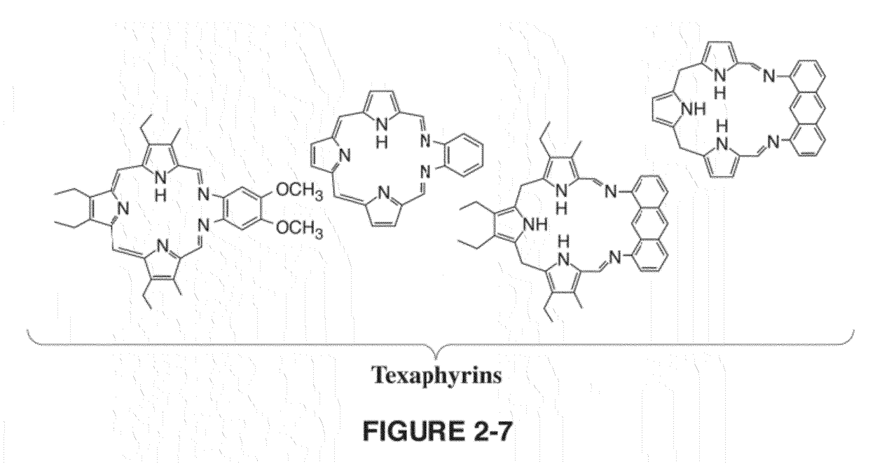

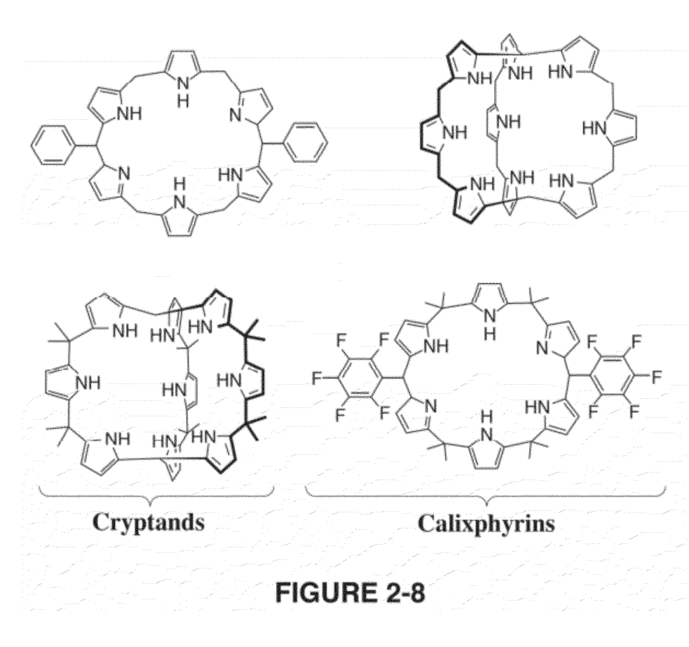

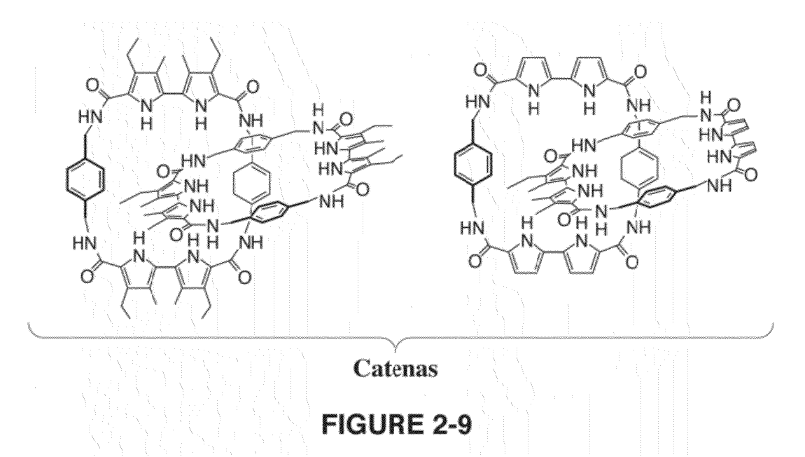

While FIG. 1 shows non-limiting examples of macrocycles comprising four nitrogen containing rings fused together, those of ordinary skill in the art will understand and know of macrocycles comprising more than four fused rings, for example, five, six, or seven fused rings (e.g., expanded macrocycles). Non-limiting examples of expanded macrocycles comprising more than four fused rings which may be used in connection with the present invention include porphycenes, [18]porphyrins(2.1.0.1), N-confused porphyrins, sapphyrins (e.g., functionalized and unfunctionalized), heterosapphyrins, rubyrins, orangarins, cycle[8]pyrroles, rosarins, turcasarins, texaphyrins, cryptans, calixphyrins, and catenanes. Non-limiting examples of expanded macrocycles are shown in FIG. 2, wherein each may be substituted with a pendant group as described herein, and/or one or more beta-positions may be substituted, optionally with an electron-withdrawing group.

In some embodiments, a macrocycle comprises at least one pendant group (e.g., Y). The term “pendent group,” as used herein (e.g., “Y”), refers to a group which is of substantial length and of appropriate orientation and/or rigidity to allow for a portion of the pendant group or a substituent on the pendant group to be oriented over the face of the macrocycle. Such an orientation may be an important feature for the application of these compounds (e.g., in catalysis). Those of ordinary skill in the art will be able to select appropriate pendent groups of suitable length and/or rigidity. In some cases, the pendent group is selected from alkyl, heteroaryl, cycloalkyl, heterocyclolkyl, aryl, alkylaryl, arylalkyl, alkoxy, amino, heteroalkyl, each optionally substituted. In some cases, the pendent group comprises a plurality of fused aryl, heteroaryl, cycloalkyl, and/or heterocycloalkyl rings, each optionally substituted. In some cases, a pendent group comprises xanthene, dibenzofuran, biphenylene, or anthracene, each optionally substituted. In some cases, the pendant group comprises the structure:

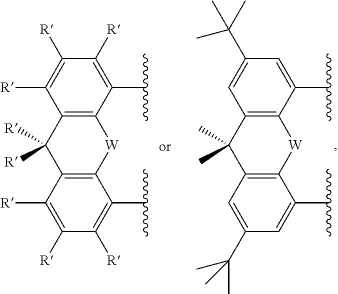

wherein W is a heteroatom and one [symbol] indicates a connection to the macrocycle and the other [symbol] indicates H or an optional substituent (e.g., -G, or —Z—P_{g}, or -Z-D_{g}, or —Z—H, as described herein), and wherein each R¹ is independently hydrogen, alkyl, aryl, heteroalkyl, and heteroaryl, each optionally substituted. In some cases, W is O, S, or NR, wherein R is a suitable substituent (e.g., H, alkyl, aryl, etc., each optionally substituted).

In some cases, pendant group Y is optionally substituted, e.g., such that it comprises the formula -Y-G, or —Y—Z—P_{g}, or -Y-Z-D_{g}, or —Y—Z—H, wherein Z, G, P_{g}, and D_{g} are described herein, and Y is as described above. For example, Y may be alkyl, heteroaryl, cycloalkyl, heterocyclolkyl, aryl, alkylaryl, arylalkyl, alkoxy, amino, or heteroalkyl, each optionally substituted with —Z—P_{g}, or -Z-D_{g}, or —Z—H, or -G.

In some embodiments, Z is a hydrolysable group. The term “hydrolyzable group,” as used herein, refers to a group which is able to be hydrolyzed under selected conditions. For example, a hydrolyzable group, in some embodiments, comprises a protecting group (e.g., —Z—P_{g}), and may be hydrolyzed (e.g., to form —Z—H or -Z-D_{g}) under appropriate conditions. Generally, a hydrolyzable group is associated with a protecting group via a covalent bond. Non-limiting examples of hydrolyzable groups comprising a protecting group (e.g., —Z—P_{g}) include —COOP_{g}, —PO(OR)(OP_{g}), —B(OR)(OP_{g}), —CO(NR)(NP_{g}), —NRP_{B}, —C(NR₂)(NRP_{g}), —OP_{g}, and the like, wherein R is any suitable substituent as will be known by those of ordinary skill in the art (e.g., R is alkyl, aryl, heteroalkyl, heteroalkyl, etc., each optionally substituted) and P_{g} is a protecting group. Protecting groups are described herein.

In some embodiments, a pendent group may be substituted with -Z-D_{g}, wherein Z is as described herein (e.g., a hydrolyzable group) and D_{g} is a deprotected group or optionally absent. The term “deprotected group,” as used herein, refers to a group which can be readily replaced with a hydrogen. In some cases, the deprotected group is associated with the hydrolyzable group via a ionic bond (e.g., -Z-D_{g} may be —[COO⁻][K⁺]). A non-limiting example of a deprotected group is a cation (e.g., K⁺, Na⁺). For example, in some cases, the hydrolyzable group may be selected such that following exposure of a compound having the formula X—Y—Z—P_{g} to microwave energy for a sufficient period of time, a compound having the formula X-Y-Z-D_{g} forms, wherein D_{g} is a deprotected group or optionally absent. Non-limiting example of -Z-D_{g} are -Z-D_{g} is —COOD_{g}, —PO(OR)(OD_{g}), —B(OR)(OD_{g}), —CO(NR)(ND_{g}), —NRD_{g}, —C(NR₂)(NR)D_{g}), and —OD_{g}, wherein R is a suitable organic substituent as will be known by those of ordinary skill in the art (e.g., R is alkyl, aryl, heteroalkyl, heteroalkyl, etc., each optionally substituted), and X, Y, and Z are as described herein.

In some embodiments, a pendent group may be substituted with —Z—H, wherein Z is as described herein. For example, as described herein, in some cases, a compound having the formula X-Y-Z-D_{g} may be further reacted to form a compound having the formula X—Y—Z—H. Non-limiting examples of —Z—H include —COOH, —PO(OR)(OH), —B(OR)(OH), —CO(NR)(NH), —NRH, —C(NR₂)(NR)H, and —OH).

In some embodiments, a compound may further comprise at least one linker group, L, between the pendant group Y and a hydrolyzable group. For example, a compound may comprise the formula X-Y-L-Z—P_{g}, or X-Y-L-Z-D_{g}, or X-Y-L-Z—H. A linker group may be included to provide proper spacing and/or orientation of a group (e.g., P_{g}, D_{g}, H) over the center of the macrocycle. Non-limiting examples of linker groups include alkyl, aryl, heteroalkyl, heteroaryl, alkylaryl, arylalkyl, etc., each optionally substituted.

In some embodiments, Z is —COO— such that —Y—Z—P_{g} has the formula —Y—COOP_{g}, or such that Y-Z-D_{g} has the formula —Y—COOD_{g}, or such that Y—Z—H has the formula —Y—COOH, wherein P_{g} and D_{g} are as described herein. It should be understood, while much of the discussion herein focuses on a hydrolyzable group (e.g., Z) comprising the formula —COO—, this is by no means limiting and other hydrolyzable groups may be used, for example, an amino group.

In some embodiments, —Y—Z—P_{g} comprises the formula:

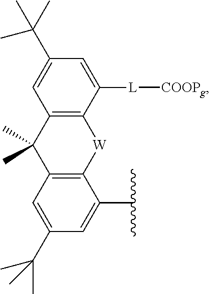

wherein W is a heteroatom (e.g., O, NR, S, etc., wherein R is a suitable substituent, e.g., H, alkyl, aryl, etc., each optionally substituted), L is a linker group, optionally present (e.g., alkyl, aryl, heteroalkyl, heteroaryl, etc. each optionally substituted) and P_{g} is a protecting group, as described herein. In some cases, W is O. As will be understood by those of ordinary skill in the art, P_{g} in this structure may optionally be substituted for any other substituent described herein (e.g., -D_{g}, —H)

The term “protecting group,” as used herein, (e.g., “P_{g}”) includes any suitable protecting group as will be known to and understood by those of ordinary skill in the art. “Protected form” refers to a substituent in which an atom such as hydrogen has been removed and replaced with a corresponding protecting group. Generally, the protecting group is susceptible to being removed or replaced by exposure to microwave irradiation (e.g., in the presence of a base), as described herein. In some cases, P_{g} is selected from the group consisting of alkyl, aryl, heteroalkyl, or heteroaryl, each optionally substituted, and Y is a pendent group as described herein. In some cases, P_{g} is methyl, ethyl, propyl, isopropyl, butyl, or other alkyl groups, and benzyl, or other aryl groups, each optionally substituted. Other non-limiting examples of protecting groups include, but are not limited to, carboxy protecting groups (for producing the protected form of carboxylic acid); amino-protecting groups (for producing the protected form of amino); sulfhydryl protecting groups (for producing the protected form of sulfhydryl); etc. Particular examples include but are not limited to: benzyloxycarbonyl, 4-nitrobenzyloxycarbonyl, 4-bromobenzyloxycarbonyl, 4-methoxybenzyloxycarbonyl, methoxycarbonyl, tert-butoxycarbonyl, isopropoxycarbonyl, diphenylmethoxycarbonyl, 2,2,2-trichloroethoxycarbonyl, 2-(trimethylsilyl)ethoxycarbonyl, 2-furfuryloxycarbonyl, allyloxycarbonyl, acetyl, formyl, chloroacetyl, trifluoroacetyl, methoxyacetyl, phenoxyacetyl, benzoyl, methyl, t-butyl, 2,2,2-trichloroethyl, 2-trimethylsilyl ethyl, 1,1-dimethyl-2-propenyl, 3-methyl-3-butenyl, allyl, benzyl, para-methoxybenzyldiphenylmethyl, triphenylmethyl (trityl), tetrahydrofuryl, methoxymethyl, methylthiomethyl, benzyloxymethyl, 2,2,2-trichloroethoxymethyl, 2-(trimethylsilyl)ethoxymethyl, methanesulfonyl, para-toluenesulfonyl, trimethylsilyl, triethylsilyl, triisopropylsilyl, acetyl (Ac or —C(O)CH₃), benzoyl (Bn or —C(O)C₆H₅), and trimethylsilyl (TMS or —Si(CH₃)₃), and the like; formyl, acetyl, benzoyl, pivaloyl, t-butylacetyl, phenylsulfonyl, benzyl, t-butyloxycarbonyl (Boc), and benzyloxycarbonyl (Cbz) and the like; and hemithioacetals such as 1-ethoxyethyl and methoxymethyl, thioesters, or thiocarbonates and the like.

In some embodiments, the pendant group (e.g., Y) is substituted with -G, wherein G is a substituent which is capable of coordinating with a metal atom or a semi-metal atom, but is not necessarily hydrolyzable (e.g., as is described above for Y). For example, G may comprise an element comprising lone pair of electrons (e.g., an amidine, an alcohol), which is capable of interacting with a metal or a semi-metal positioned within the central binding cavity of the macrocycle. Non-limiting examples of G groups include amidines, alcohols, amines, amides, heteroaryls, sulfones, and sulfides. In some cases, a linking group, L, may be optionally present between Y and G (e.g., —Y-L-G). Non-limiting examples of suitable L groups are described herein. In some cases, G may be a cationic group. In some cases, the crown molecule may be associated with an cation (e.g., Li⁺).

In some embodiments, a macrocycle as described herein may comprise one more groups which aid in the solubility of the macrocycle. In some cases, the macrocycle may comprise one or more groups which increases the water solubility of the macrocycle. For example, the at least one water-soluble group may be present at at least one R¹ or at least one R⁵ as shown in FIG. 1. In some cases, the water-soluble group may be an ionic group. The term ionic group is given its ordinary meaning in the art and refers to anionic groups, cationic groups, and groups (sometimes referred to as “ionogenic” groups) that are uncharged in one form but can be easily converted to ionic groups (for example, by protonation or deprotonation in aqueous solution). Non-limiting examples of ionic groups include, but are not limited to, carboxylate, sulfonate, phosphate, amine, N-oxide, ammonium (e.g., including quaternized heterocyclic amines such as imidazolium and pyridinium) groups, uronic acids, carboxylic acid, sulfonic acid, amine, and moieties such as guanidinium, phosphoric acid, phosphonic acid, phosphatidyl choline, phosphonium, borate, sulfate, etc. Other non-limiting examples of water-solubilizing groups include butanenitrile, 4-[[(1,1-dimethylethyl)dimethylsilyl]oxy]-2-[2-[[(1,1-dimethylethyl)dimethylsilyl]oxy]ethyl]-; butanal, 4-[[(1,1-dimethylethyl)dimethylsilyl]oxy]-2-[2-[[(1,1-dimethylethyl)dimethylsilyl]oxy]ethyl]-; acetic acid, 2-(4-formylphenoxy)-, 1,1-dimethylethyl ester; acetic acid, (4-formylphenoxy)-, 1,1-dimethylethyl ester (9CI); (4-formylphenoxy)acetic acid 1,1-dimethylethyl ester; 4-[[(tert-butoxycarbonyl)methyl]oxy]benzaldehyde; 4-tert-butoxycarbonylmethoxybenzaldehyde; tert-butyl (4-formylphenoxy)acetate; 1,5-pentanediol, 3-(di-1H-pyrrol-2-ylmethyl)-; phosphoric acid, P,P′-[3-(di-1H-pyrrol-2-ylmethyl)-1,5-pentanediyl] P,P,P′,P′-tetramethyl ester; phosphoric acid, 3-(di-1H-pyrrol-2-ylmethyl)-1,5-pentanediyl tetramethyl ester (9CI); phosphoric acid, 3-(di-1H-pyrrol-2-ylmethyl)-5-hydroxypentyl dimethyl ester; acetic acid, 2-[4-[bis(5-formyl-1H-pyrrol-2-yl)methyl]phenoxy]-, 1,1-dimethylethyl ester; acetic acid, [4-[bis(5-formyl-1H-pyrrol-2-yl)methyl]phenoxy]-, 1,1-dimethylethyl ester (9CI); (dimethoxyphosphinyl)oxy]ethyl]propyl-; (dimethoxyphosphinyl)oxy]ethyl]propyl; [2-(dimethoxyphosphinyl)ethyl]propyl]; 2-(dimethoxyphosphinyl); [3-[[(1,1-dimethylethyl)dimethylsilyl]oxy]-1-[2-[[(1,1-dimethylethyl)dimethylsilyl]oxy]ethyl]propyl]-; [[(1,1-dimethylethyl)dimethylsilyl]oxy]-1-[2-[[(1,1-dimethylethyl)dimethylsilyl]oxy]ethyl]propyl]. Those of ordinary skill in the art will be aware of other suitable water-solubilizing group, for example, as described in International Patent Application Publication No. WO/2007/047925, published Apr. 26, 2007, herein incorporated by reference. In some cases, increased water solubility of a macrocycle may be useful in embodiments where the macrocycle is being employed in homogeneous catalysts (e.g., as described herein).

Those of ordinary skill in the art will be aware of methods and techniques for forming suitable macrocycles having the formula X-Y. For example, as shown in Equation 1, a substituted porphyrin may be synthesized by reaction of at least one aldehyde and at least one pyrrole (e.g., optionally substituted), using Lindsey conditions (e.g., acid catalyzed condensation the reaction component), wherein R¹, R⁵, and M are as described herein.

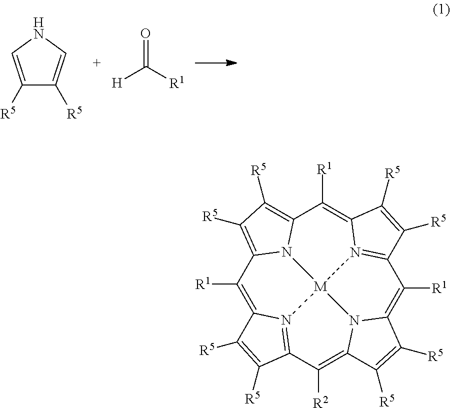

In some cases, each or at least one R⁵ of the pyrrole is an electron-withdrawing group (e.g., halide). In some cases, at least one R⁵ of the pyrrole is fluorine. In some cases, both R⁵ groups of the pyrrole are fluorine.

Those of ordinary skill in the art will understand that the ratio of the aldehyde to pyrrole provided in a reaction may be adjusted to optimized reaction conditions, and/or that more than one type of aldehyde and/or pyrrole may be provided to the reaction mixture such that the substitution about the formed porphyrin may be varied. For example, in some cases, a first type of aldehyde and a second type of aldehyde may be provided, wherein R¹ of the first a type of aldehyde is a suitable substituent (e.g., hydrogen, alkyl, aryl, heteroalkyl, heteroaryl, alkenyl, alkynyl, cycloalkyl, cycloalkylalkyl, cycloalkylalkenyl, cycloalkylalkynyl, acyl, carboxylic acid, acylamino, alkylthio, amino, alkylamino, arylalkylamino, arylalkyl, alkylaryl, and alkoxy, each optionally substituted), and R¹ of the second type of aldehyde differs from R¹ of the first type of aldehyde and comprises Y, optionally substituted (e.g., such that the porphyrin formed comprises at least one pendent group). For example, in some cases, at least one R¹ is Y, as shown in Equation 2:

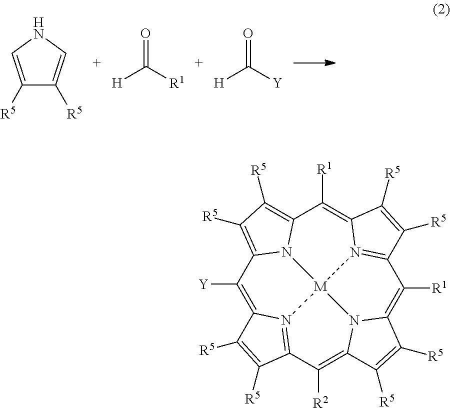

In some cases, more than one R¹ is Y. For example, as shown in Equation 3, two R¹ are Y.

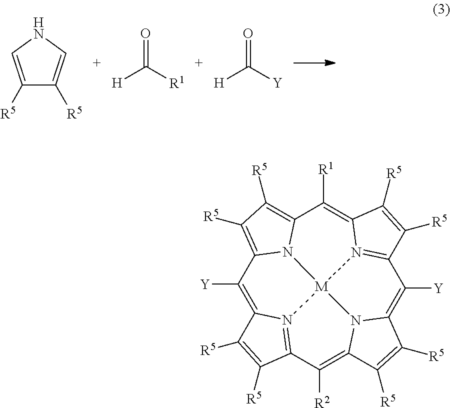

In Equation 3, the trans isomer is shown. As described herein, in some cases, the cis isomer may form or a combination of the cis and trans isomers move form. Each Y and each R¹ can be the same or different. For example, in some cases, each R¹ are the same. In other cases, each R¹ is different. In some cases, each Y is the same. In some cases, each Y is different.

The ratio of the first type of aldehyde and the second type of aldehyde may be adjusted, such that the porphyrin formed comprises the desired number of each type of aldehyde. In some cases, the ratio of the first type of aldehyde to the second type of aldehyde provided is about 20:1, about 15:1, about 10:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, or about 1:20.

Similarly, more than one type of pyrrole may be provided. For example, a first type of pyrrole and a second type of pyrrole may be provided, wherein the R⁵ of the second type of pyrrole differs from the R⁵ of the first type of pyrrole. In some cases, each R⁵ of the first type of pyrrole is H and at least one R⁵ of the second type of pyrrole is not H (e.g., each R⁵ can be the same or different and is hydrogen, halide, CN, NO₂, alkyl, aryl, heteroalkyl, heteroaryl, alkenyl, alkynyl, cycloalkyl, cycloalkylalkyl, cycloalkylalkenyl, cycloalkylalkynyl, acyl, carboxylic acid, carboxylate, OH, acylamino, alkylthio, amino, alkylamino, arylalkylamino, or alkoxy, each optionally substituted). In some cases, R⁵ of the first type of pyrrole is H and at least one R⁵ of the second type of pyrrole is halide (e.g., F). In some cases, at least one or both R⁵ of the first type of pyrrole is Cl, Br, or I and at least one or both R⁵ of the second type of pyrrole is F.

The ratio of the first type of pyrrole and the second type of pyrrole may be adjusted, such that the porphyrin formed comprises the desired number of each type of pyrrole. In some cases, the ratio of the first type of pyrrole to the second type of pyrrole is about 20:1, about 15:1, about 10:1, about 5:1, about 4:1, about 3:1, about 2:1, about 1:1, about 1:2, about 1:3, about 1:4, about 1:5, about 1:6, about 1:7, about 1:8, about 1:9, about 1:10, about 1:15, or about 1:20.

As will be understood by those of ordinary skill in the art, while much of the discussion provided herein regarding synthesis is related to macrocycles comprising porphyrins, this is by no means limiting, and those of ordinary skill in the art will be able to apply the methods and teachings provided herein to other macrocycles.

In some cases, the porphyrin may be formed by providing a reaction mixture comprising the reactants, followed by addition of BF₃.OEt₂, followed by addition of 2,3-dichloro-5,6-dicyanobenzoquinone (DDQ) or another oxidant (e.g., quinine derivative, p-chloranil, air, oxygen, oxygen/phthalocyanines, non-nucleophilic bases, etc.). Those of ordinary skill in the art will be aware of other methods and techniques for synthesizing appropriate macrocycles (e.g., stepwise synthesis starting with pyrrole and an aldehyde, forming a macrocycle comprising a good leaving group (e.g., bis(pinacolato)diboron), followed by replacement of the leaving group with -Y (e.g., -Y). The Examples Section describes additional non-limiting synthetic methods for the formation of a macrocycle (e.g. comprising a pendant group), for example, see Schemes 2 and 5-7.

In some embodiments, methods are provided for producing a compound having the formula X—Y—COOH (e.g., X—Y—Z—H) from a compound having the formula X—Y—COOP_{g}, wherein P_{g} is a protection group (e.g., X—Y—Z—P_{g}). In some embodiments, the method comprises the steps indicated in Equation 4.

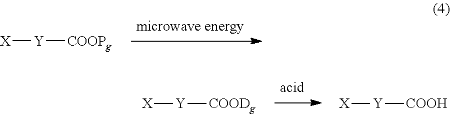

Specifically, the method may comprise providing a compound having the formula X—Y—COOP_{g}, wherein X, Y, and P_{g} are as described herein, and exposing the composition to microwave energy for a sufficient period of time to form a composition having the formula X—Y—COOD_{g}, wherein D_{g} is a deprotected group (e.g., a cationic species such as Na⁺, K⁺, etc.) or optionally absent, as described herein. X—Y—COOD_{g} may be further reacted (e.g., with an acid) to form a compound having the formula X—Y—COOH.

In some cases, the exposing step comprises subjecting a solution comprising a compound having the formula X—Y—Z—P_{g} (e.g., X—Y—COOP_{g}) to microwave energy in the presence of at least one additive. Solvents and reactions conditions are described herein. In some cases, at least one additive may be present in the solution during the exposing step. In some instances, the at least one additive is a base. Without wishing to be bound by theory, the presence of a base may aid in the hydrolysis of the ester functional group. Non-limiting examples of appropriate bases include NaOH, KOH, and the like. The at least one additive (e.g., base) may be present in a suitable concentration, for example, at about 1 N, about 2 N, about 3 N, about 4 N, about 5 N, about 6 N, about 7 N, about 8 N, about 9 N, about 10 N, or greater. Simple screening tests may be used to determine an optimal concentration of base (e.g., minimize side product, maximize conversion). In some cases, the base may be present at a concentration of about 6 N.

The compound having the formula X-Y-Z-D_{g} (e.g., X—Y—COOD_{g} formed by exposing a compound having the formula X—Y—COOP_{g} to microwave energy) may then be exposed to an acid, thereby forming a compound having the formula X—Y—Z—H (e.g., X—Y—COOH). The protonation of the compound may proceed by providing an acid to a solution comprising the compound X-Y-Z-D_{g} (e.g., X—Y—COOD_{g}). Non-limiting examples of suitable acids include HCl, HBr, trifluoroacetic acid, sulfuric acid, para-toluenesulfuric acid, etc. The acid may be provided as a solution (e.g., aqueous solution). The compound X-Y-Z-D_{g} (e.g., X—Y—COOD_{g}) may be exposed to the acid for any suitable time period, for example, at least about 30 minutes, at least about 1 hour, at least about 2 hours, at least about 4 hours, at least about 8 hours, at least about 12 hours, at least about 16 hours, at least about 18 hours, at least about 24 hours, at least about 48 hours, or greater, or between about 1 hour and about 48 hours, between about 1 hour and about 24 hours, between about 2 hours and about 18 hours, or between about 4 hours and about 16 hours. The compound may be exposed to the acid, in some cases, for a period of time such that conversion of the compound into X—Y—Z—H (e.g., X—Y—COOH) is substantially complete (e.g., as determine using a method known to those of ordinary skill in the art, e.g., thin layer chromatography).

In some embodiments, additional steps may be conducted prior to and/or following the exposing step. For example, a reaction intermediate (e.g., X-Y-Z-D_{g} such as X—Y—COOD_{g}) or a reaction product (e.g., X—Y—Z—H such as X—Y—COOH) may be isolated (e.g., via distillation, column chromatography, extraction, etc.) and/or analyzed (e.g., gas liquid chromatography, high performance liquid chromatography, nuclear magnetic resonance spectroscopy, etc.) using commonly known techniques.

In some embodiments, the present invention provides methods for metallating a macrocycle. The mixture comprising a macrocycle and a metal complex comprising a metal atom (or a semi-metal) may be exposed to microwave energy, thereby forming a compound comprising the macrocycle and the metal atom, wherein the metal atom is coordinated by at least some of the heteroatoms within the central binding cavity of the macrocycle.

In some cases, a method comprises forming a mixture of a metal complex comprising a metal atom and a composition having the formula:

X-Y;

wherein X-Y is as described herein (e.g., wherein X is a macrocycle, Y is a pendent group, optionally substituted, and wherein at least one beta-position of the macrocycle is an electron-withdrawing group, e.g., halide) followed by exposing the mixture to microwave energy, thereby forming a compound comprising the macrocycle and the metal atom. Generally, the metal atom is coordinated by at least some of the heteroatoms within the central binding cavity of the macrocycle. In some cases, the pendant group Y comprising a least one substituent (e.g., —Z—P_{g}, -Z-D_{g}, or —Z—H). In some cases, X comprises a macrocycle comprising heteroatoms positioned such that at least some of the heteroatoms are able to coordinate a metal or semi-metal within a central binding cavity of the macrocycle. In some embodiments, the macrocycle comprised in the mixture has one or more hydrogen atoms coordinated to the heteroatoms within the central binding cavity of the macrocycle. In some cases, at least a portion of the pendent group may coordinate with the metal atom or semi-metal atom

As noted above, in some embodiments, the macrocycles comprises more than one pendant group. In such embodiment, the metal atom may be coordinated by all, a portion of, or only one of the pendant groups. In some cases, the interaction between a metal atom and a pendant group may depend, at least in part, on the geometric and steric conditions of the compound comprising the macrocycle and the metal atom.

Non-limiting example of metals include alkali metals (e.g., lithium, sodium, potassium), alkaline earth metals (e.g., magnesium, calcium), transition metals (e.g., chromium, manganese, titanium, vanadium, iron, cobalt, nickel, copper, zinc, and third and fourth row transition metals), and lanthanides. In some embodiments, M may be a semi-metal (e.g., boron, silicon, germanium, arsenic, antimony, tellurium). In a particular embodiment, the metal atom is cobalt or iron. Those of ordinary skill in the art will be able to select appropriate metal complexes to be provided to the mixture. For example, the metal complex may comprise the metal atom and a plurality of ligands, wherein the ligands are susceptible to dissociation. Non limiting examples of suitable ligands include acetate and halides.

In some cases, for the metallation reactions, the heteroatoms of the macrocycle prior to exposure to microwave energy may be associated with one or more hydrogen atoms. At least one of the hydrogen atoms may be substituted by the metal atom. In some cases, all of the hydrogen atoms (e.g., one, two, three, or four) may be substituted by a single metal atom (e.g., wherein the metal atom is associated with all of the heteroatoms). For example, when the macrocycle is a porphyrin, the four nitrogen atoms of the macrocycle are generally associated with two hydrogen atoms. At least one of the two hydrogen atoms may be replaced by a metal atom.

In some cases, both hydrogen atoms are replaced by the metal atom. In some embodiments, at least one auxiliary ligand may be associated with the metal following association of the metal with the macrocycle. The ligand may be in an apical position with respect to the macrocycle. An auxiliary ligand may or may not be charged. Non-limiting examples of auxiliary ligands include halides (e.g., chlorine, fluorine, bromine, iodine), cysteine, and coordinating solvents (e.g., pyridine, tetrahydrofuran, diethyl ether, indoles and derivatives, imidazole, and derivatives etc.).

In some embodiments, the association between a metal atom and at least some of the heteroatoms of the macrocycle may comprise the formation of at least one bond between the metal atom and at least one heteroatom of the macrocycle. Non-limiting examples of types of bond include an ionic bond, a covalent bond (e.g. carbon-carbon, carbon-oxygen, oxygen-silicon, sulfur-sulfur, phosphorus-nitrogen, carbon-nitrogen, metal-oxygen or other covalent bonds), a hydrogen bond (e.g., between hydroxyl, amine, carboxyl, thiol and/or similar functional groups, for example), a dative bond (e.g. complexation or chelation between metal ions and monodentate or multidentate ligands), Van der Waals interactions, and the like. “Association” of a metal atom with at least one heteroatom would be understood by those of ordinary skill in the art based on this description. In some cases, the association comprises the formation of at least one ionic bond and/or at least one dative bond.

Those of ordinary skill in the art will be able to determine suitable conditions under which to expose a solution (e.g., comprising a macrocycle, or comprising a mixture comprising a macrocycle and a metal complex) to microwave energy. Conditions which may be varied include, but are not limited to, time of exposure, power of the microwave energy, pressure, duration time (e.g., time required to reach the selected conditions), solvent, additives, and temperature. Those of ordinary skill in the art will be aware of systems that may be used for applying microwave energy to a solution. The term “microwave energy,” as used herein, is given its ordinary meaning in the art and refers to electromagnetic waves with wavelengths ranging from between about one meter to about one millimeter.

In some embodiments, the temperature of the reaction mixture at which the exposing step (e.g., to microwave energy) is conducted may be varied. As will be understood by those of ordinary skill in the art, generally, at lower temperatures, a reaction proceeds at a slower rate as compared to a higher temperature, however, the amount of side products produced generally increases at higher temperatures. Using simple screening tests, those of ordinary skill in the art will be able to select an appropriate temperature(s) for exposing a solution to microwave energy. In some embodiments, the exposing step is conducted at room temperature, that is, between about 15° C. and about 25° C., between about 18° C. and about 22° C., or at about 20° C. In some cases, the exposing step may be conducted at temperatures greater than room temperature. For example, the temperature may be at least about 30° C., at least about 40° C., at least about 50° C., at least about 60° C., at least about 70° C., at least about 80° C., at least about 90° C., at least about 100° C., or greater. In a particular embodiment, the temperature is between about 60° C. and about 80° C., or between about 65° C. and about 75° C., or at about 70° C., or between about 50° C. and about 70° C., or between about 55° C. and about 65° C., or at about 60° C.

A solution may be exposed to microwave energy for any suitable period of time. In some embodiments, the length of the exposing step is determined by whether a substantial portion of the starting material has been transformed into the desired product, for example, by using simple screening tests known to those of ordinary skill in the art. For example, a small amount of the reaction mixture may be analyzed using thin layer chromatography. In some cases, a solution is exposed to microwave energy for about 1 minute, about 2 minutes, about 3 minutes, about 5 minutes, about 10 minutes, about 15 minutes, about 20 minutes, about 30 minutes, about 45 minutes, about 60 minutes, about 2 hours, about 4 hours, about 8 hours, about 12 hours, about 18 hours, about 24 hours, or greater. In some cases, the period of time is between about 1 minute and about 24 hours, between about 1 minute and about 12 hours, between about 1 minute and about 6 hours, between about 1 minute and about 2 hours, between about 1 minute and about 15 minutes, between about 5 minutes and about 30 minutes, between about 5 minutes and about 15 minutes, or the like.

The microwave energy may be applied at any suitable power and/or intensity. In some embodiments, the microwave energy may be applied at a transmit power level of no more than about 5 W, about 10 W, about 15 W, about 20 W, about 50 W, about 100 W, about 200 W, about 400 W, about 500 W, about 750 W, or about 1000 W. In some embodiments, the microwave energy may be applied at a transmit power level of between about 1 W and about 1000 W, between about 1 W and about 500 W, between about 1 W and about 300 W, between about 1 W and about 100 W, between about 1 W and about 50 W, between about 1 W and about 30 W, between about 1 W and about 10 W, or between about 1 W and about 5 W. In certain embodiments, the power density may be no more than about 5 W/m², about 10 W/m², about 15 W/m², about 20 W/m², about 50 W/m², about 100 W/m², about 200 W/m², about 400 W/m², about 500 W/m², about 750 W/m², or about 1000 W/m². In certain embodiments, the power density may be between about 1 W/m² and about 1000 W/m², between about 1 W/m² and about 500 W/m², between about 1 W/m² and about 300 W/m², between about 1 W/m² and about 100 W/m², between about 1 W/m² and about 50 W/m², between about 1 W/m² and about 30 W/m², between about 1 W/m² and about 10 W/m², or between about 1 W/m² and about 5 W/m².

The solution to which microwave energy is applied may comprise one or more solvents. In some embodiments, the solvent is chosen such that the starting materials are at least partially soluble. Non limiting examples of suitable solvents include tetrahydrofuran, acetonitrile, dimethylformamide, chloroform, dichloromethane, methanol, toluene, hexanes, xylene, diethyl ether, glycol, dioxane, dimethylsulfoxide, ethyl acetate, pyridine, triethylamine, or combinations thereof (e.g., 10:1 chloroform:methanol). In some cases, the methods described herein may be performed in the absence of solvent (e.g., neat).

Those of ordinary skill in the art will be aware of equipment and methods for exposing a reaction mixture and/or solution to microwave energy. In some embodiments, the system may be equipped with various sensors for monitoring the synthesis, for example, pressure, power, and temperature sensors. Microwave energy may be produced using any suitable source of microwave energy, including many commercially-available sources. For instance, microwave energy may be produced using microwave applicators (which may be handheld in some cases), vacuum tube-based devices (e.g., a magnetron, a klystron, a traveling-wave tube, a gyrotron), certain field-effect transistors or diodes (e.g., tunnel diodes, Gunn diodes), or the like.

In some embodiments, the methods may be carried out in a sealed reaction container, for example a crimp-sealed thick-walled glass tube. In some embodiments, the reaction mixture may be agitated during exposure to microwave energy. For example, the reaction mixture may be stirred (e.g., using a magnetic stir bar) or shaken.

The pressure during exposure to microwave energy may also be varied. The reaction may be carried out at about atmospheric pressure, or in some cases, may be carried out above atmospheric pressure. In some cases, an increase in pressure of the reaction may be caused, at least in part, because the reaction is being carried out in a sealed system. For example, in embodiments where the reaction is carried out in a sealed system, the pressure of the reaction may increase upon heating of the reaction mixture. In some cases, the reaction may be carried out at pressures between about 1 atm and about 30 atm, between about 1 atm and about 20 atm, between about 5 atm and about 20 atm, or at about 1 atm, about 2 atm, about 3 atm, about 5 atm, about 10 atm, about 15 atm, about 20 atm, about 25 atm, about 30 atm, or greater.

In some embodiments, a reaction product (e.g., X-Y, optionally having Y substituted (e.g., -Y-G, —Y—Z—P, -Y-Z-D_{g}, —Y—Z—H), optionally comprising a coordinated metal atom) may be isolated, for example, using extraction techniques. A product may be isolated with an improved yield as compared to previously known techniques. In some cases, a product may be isolated with a yield between about 10% and about 50%, between about 15% and about 40%, or between about 20% and about 40%. In some cases, a product may be isolated with a yield of at least about 15%, at least about 20%, at least about 25%, at least about 30%, at least about 35%, at least about 40%, at least about 50%, at least about 60%, at least about 70%, at least about 80%, at least about 90%, or the like.

The compositions as described herein, and compositions formed using the methods described herein, may find various applications, for example, for use as catalysts. In some cases, the composition comprises a metal coordinated by the macrocycle (e.g., wherein M is a metal or a semi-metal). In some cases, the composition comprises the formula:

X-Y

wherein X comprising a macrocycle (e.g., as described herein, for example, having 2-7 heteroatoms positioned such that at least some of the heteroatoms are able to coordinate a metal within a central binding cavity of the macrocycle) and Y is a pendent group, optionally substituted. At least one beta-position of the macrocycle may or may not be an electron-withdrawing group (e.g., halide). In some cases, at least one beta-position of the macrocycle is an electron-withdrawing group (e.g., halide).

In some embodiments, a method of catalysis is provided comprising providing a composition having the formula:

X-Y;

wherein X comprises a macrocycle having 2-7 heteroatoms positioned such that at least some of the heteroatoms are able to coordinate a metal within a central binding cavity of the macrocycle, wherein at least one beta-position of the macrocycle is an electron-withdrawing group; and Y is a pendent group, optionally substituted, wherein at least one metal atom is coordinated by the macrocycle and/or the pendent group; and exposing the composition to a reactant, wherein a product is formed from the reactant following application of a voltage to the composition.

In some embodiments, a method forming oxygen gas from water is provided comprising providing a composition, having the formula:

X-Y;

wherein X comprises a macrocycle having 2-7 heteroatoms positioned such that at least some of the heteroatoms are able to coordinate a metal within a central binding cavity of the macrocycle, wherein at least one beta-position of the macrocycle is an electron-withdrawing group; and Y is a pendent group, optionally substituted, wherein at least one metal atom is coordinated by the macrocycle and/or the pendent group; and exposing the composition to water, wherein oxygen gas is formed from water following application of a voltage to the composition.

In some embodiments, a method of forming hydrogen gas from water is provided comprising providing a composition, having the formula:

X-Y;

wherein X comprises a macrocycle having 2-7 heteroatoms positioned such that at least some of the heteroatoms are able to coordinate a metal within a central binding cavity of the macrocycle; and Y is a pendent group, optionally substituted, wherein at least one metal atom is coordinated by the macrocycle and/or the pendent group; and exposing the composition to water, acid, organic solvent, or combination thereof, wherein hydrogen gas is formed from the water, acid, organic solvent, or combination thereof following application of a voltage to the composition.

In some cases, a method of reducing CO₂ is provided comprising providing a composition, having the formula:

X-Y;

wherein X comprises a macrocycle having 2-7 heteroatoms positioned such that at least some of the heteroatoms are able to coordinate a metal within a central binding cavity of the macrocycle, wherein at least one beta-position of the macrocycle is an electron-withdrawing group; and Y is a pendent group, optionally substituted, wherein at least one metal atom is coordinated by the macrocycle and/or the pendent group; and exposing the composition to CO₂, wherein the CO₂ is reduced following application of a voltage to the composition.

Those of ordinary skill in the art will be aware of methods and techniques for forming an electrode comprising a composition as described herein. In some cases, a solution may be formed comprising the composition and a solvent. A substrate (e.g., comprising a conductive material, such as FTO coated glass) may be exposed to the solution. The solvent may be evaporated (e.g., in air, using vacuum, and/or using heat), and the composition may form a coating on the substrate (e.g., as a film), thereby forming the electrode comprising the substrate and a layer of the composition. Following formation of the electrode, the electrode may be employed in a wide variety of electrochemical reactions using techniques and systems known to those of ordinary skill in the art.

In some embodiments, the compositions described herein may be employed in homogeneous catalysts systems. In some cases, the compositions is soluble and/or water-stable. Generally, the composition comprises enough redox potential to interact with substrate(s) of interest (e.g., for reaction). For example, a solution may be formed comprising a composition as described herein and a substrate for catalysis (e.g., water, acids, organic substrate, CO₂, etc.). The composition may be dissolved in a solvent (e.g. water, optionally neat) or the substrate may be a co-solvent with concentration ranges between several ppm up to 100 percent. The substrate may be introduced as a secondary phase (e.g. dissolved gas or solid) in concentrations ranging from about 0.01 nM to about 1000 mM (e.g., about 0.01 nM, about 0.1 nM, about 1 nM, about 10 nM, about 100 nM, about 1 uM, about 10 uM, about 100 uM, about 1 mM, about 10 mM, about 100 mM, about 1000 mM, or at a range between any of the listed concentrations). The concentration of the catalytic composition may range in concentrations ranging from about 0.01 nM to about 1000 mM (e.g., about 0.01 nM, about 0.1 nM, about 1 nM, about 10 nM, about 100 nM, about 1 uM, about 10 uM, about 100 uM, about 1 mM, about 10 mM, about 100 mM, about 1000 mM, or at a range between any of the listed concentrations). Activation may be achieved by application of a voltage using any standard conditions (e.g., using an electrode in a quiescent system, under dynamic control of solvent flow, etc.). Activation of the composition may also be achieved using a sacrificial oxidant (e.g. cerium(IV)) or using a photo-oxidant added to the composition solution.

In some embodiments, a composition as described herein may be used as a catalyst for water oxidation. That is, a composition of the present invention may catalyze the production of oxygen and/or hydrogen gases (e.g., from water, acid, organic solvent, or combination thereof). The water employed may have a pH between about 1 and about 14, between about 2 and about 13, between about 3 and about 12, between about 4 and about 11, between about 5 and about 10, between about 6 and about 9, or between about 6 and about 8. In some cases, the pH is about 1, about 2, about 3, about 4, about 5, about 6, about 7, about 8, about 9, about 10, about 11, about 12, about 13, or about 14.

As will be known to those of ordinary skill in the art, an example of a side reaction that may occur during the catalytic formation of oxygen gas from water is the production of hydrogen peroxide. In some cases, no or essentially no hydrogen peroxide is produced. That is, the oxygen that is in the form of hydrogen peroxide of less than about 0.01%, less than about 0.05%, less than about 0.1%, less than about 0.2%, less than about 0.3%, less than about 0.4%, less than about 0.5%, less than about 0.6%, less than about 0.7%, less than about 0.8%, less than about 0.9%, less than about 1%, less than about 1.5%, less than about 2%, less than about 3%, less than about 4%, less than about 5%, less than about 10%, etc. That is, less than this percentage of the molecules of oxygen produced is in the form of hydrogen peroxide. Those of ordinary skill in the art will be aware of methods for determining the production of hydrogen peroxide at an electrode and/or methods to determine the percentage of hydrogen peroxide produced.

In some embodiments, a composition as described herein may be used for the formation of hydrogen gas. For example, a proton source (e.g., from an acid) may be converted to hydrogen gas using a composition as described herein (e.g., comprising a macrocycle that may or may not comprise at least one beta-position being an electron-withdrawing group, e.g., halide) as a catalyst. Those of ordinary skill in the art will be aware of suitable acids which may be employed for the formation of hydrogen gas, including, but not limited to benzoic acid, and tosic acid. In some cases, an acid may have sufficient thermodynamic potential for reduction by the compositions. The acid may be provide in any suitable concentration, for example, about 0.01 mM, about 0.05 mM, about 0.1 mM, about 0.15 mM, about 0.2 mM, about 0.3 mM, about 0.4 mM, about 0.5 mM, about 1 mM, about 10 mM, about 0.1 M, about 0.5 M, about 1 M, about 2 M, about 5 M, and the like.

Those of ordinary skill in the art will be aware of other catalytic reactions in which the compositions described herein may be employed as catalysts. For example, the compounds as described herein may be employed for hydrogen peroxide decomposition, O₂ reduction, CO₂ reduction, syngas formation, and/or oxidation of organic substrates (e.g., methanol, carbon monoxide). The compounds described herein may be useful for a wide variety of applications due to the modular nature of the compounds. For example, the size, electronics, and/or sterics of the compounds may be tuned by altering the substituents of the macrocycle (e.g., R⁵ being H as opposed to F), the number, size, and composition of the pendant group (e.g., one pendant group versus two pendant groups, the length of the at least one pendant group and the proximity to the metal center, the electronic structure of the pendant group), etc.

In some cases, oxygen and/or hydrogen gases may be produced in a catalytic reaction with a turnover number of greater than about 0.01 s⁻¹, greater than about 0.05 s⁻¹, greater than about 0.1 s⁻¹, greater than about 0.2 s⁻¹, greater than about 0.3 s⁻¹, greater than about 0.4 s⁻¹, greater than about 0.5 s⁻¹, greater than about 0.6 s⁻¹, greater than about 0.7 s⁻¹, greater than about 0.8 s⁻¹, greater than about 0.9 s⁻¹, greater than about 1 s⁻¹, greater than about 5 s⁻¹, greater than about 10 s⁻¹, or greater. In some cases, the turnover number is between about 0.001 s⁻¹ and about 10 s⁻¹, between about 0.01 s⁻¹ and about 5 s⁻¹, between about 0.01 s⁻¹ and about 10 s⁻¹, between about 0.1 s⁻¹ and about 1 s⁻¹, between about 0.5 s⁻¹ and about 1.0 s⁻¹, between about 0.9 s⁻¹ and about 0.4 s⁻¹, or between about 0.5 s⁻¹ and about 0.8 s⁻¹.

In some embodiments, an electrode comprising a composition as described herein may be capable of catalytically producing oxygen gas (e.g., from water) and/or hydrogen gas with a Faradaic efficiency of about 100%, greater than about 99.8%, greater than about 99.5%, greater than about 99%, greater than about 98%, greater than about 97%, greater than about 96%, greater than about 95%, greater than about 90%, greater than about 85%, greater than about 80%, greater than about 70%, greater than about 60%, greater than about 50%, etc. The term “Faradaic efficiency,” as used herein, is given its ordinary meaning in the art and refers to the efficacy with which charge (e.g., electrons) are transferred in a system facilitating an electrochemical reaction. Loss in Faradaic efficiency of a system may be caused, for example, by the misdirection of electrons which may participate in unproductive reactions, product recombination, short circuit the system, and other diversions of electrons and may result in the production of heat and/or chemical byproducts.

U.S. Provisional Patent Application Ser. No. 61/487,217, filed May 17, 2011, entitled “METHODS AND COMPOSITIONS COMPRISING MACROCYCLES, INCLUDING HALOGENATED MACROCYCLES,” by Nocera, et al., is incorporated herein by reference.

A variety of definitions are now provided which may aid in understanding various aspects of the invention.

As used herein, the term “reacting” refers to the formation of a bond between two or more components to produce a compound. In some cases, the compound is isolated. In some cases, the compound is not isolated and is formed in situ. For example, a first component and a second component may react to form one reaction product comprising the first component and the second component joined by a covalent bond. That is, the term “reacting” does not refer to the interaction of solvents, catalysts, bases, ligands, or other materials which may serve to promote the occurrence of the reaction with the component(s).

As used herein, the term “organic group” refers to any group comprising at least one carbon-carbon bond and/or carbon-hydrogen bond. For example, organic groups include alkyl groups, aryl groups, acyl groups, and the like. In some cases, the organic group may comprise one or more heteroatoms, such as heteroalkyl or heteroaryl groups. The organic group may also include organometallic groups. Examples of groups that are not organic groups include —NO or —N₂. The organic groups may be optionally substituted, as described below.

As used herein, the term “alkyl” is given its ordinary meaning in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. An analogous convention applies to other generic terms such as “alkenyl,” “alkynyl,” and the like. Furthermore, as used herein, the terms “alkyl,” “alkenyl,” “alkynyl,” and the like encompass both substituted and unsubstituted groups.

In some embodiments, a straight chain or branched chain alkyl may have 30 or fewer carbon atoms in its backbone, and, in some cases, 20 or fewer. In some embodiments, a straight chain or branched chain alkyl has 12 or fewer carbon atoms in its backbone (e.g., C₁-C₁₂ for straight chain, C₃-C₁₂ for branched chain), has 6 or fewer, or has 4 or fewer. Likewise, cycloalkyls have from 3-10 carbon atoms in their ring structure or from 5, 6 or 7 carbons in the ring structure. Examples of alkyl groups include, but are not limited to, methyl, ethyl, propyl, isopropyl, cyclopropyl, butyl, isobutyl, tert-butyl, cyclobutyl, hexyl, cyclochexyl, and the like. In some cases, the alkyl group might not be cyclic. Examples of non-cyclic alkyl include, but are not limited to, methyl, ethyl, propyl, isopropyl, n-butyl, tert-butyl, n-pentyl, neopentyl, n-hexyl, n-heptyl, n-octyl, n-decyl, n-undecyl, and dodecyl.

The terms “alkenyl” and “alkynyl” refer to unsaturated aliphatic groups analogous in length and possible substitution to the alkyls described above, but that contain at least one double or triple bond respectively. Alkenyl groups include, but are not limited to, for example, ethenyl, propenyl, butenyl, 1-methyl-2-buten-1-yl, and the like. Non-limiting examples of alkynyl groups include ethynyl, 2-propynyl(propargyl), 1-propynyl, and the like.

The terms “heteroalkenyl” and “heteroalkynyl” refer to unsaturated aliphatic groups analogous in length and possible substitution to the heteroalkyls described above, but that contain at least one double or triple bond respectively.

The term “aryl” refers to aromatic carbocyclic groups, optionally substituted, having a single ring (e.g., phenyl), multiple rings (e.g., biphenyl), or multiple fused rings in which at least one is aromatic (e.g., 1,2,3,4-tetrahydronaphthyl, naphthyl, anthryl, or phenanthryl). That is, at least one ring may have a conjugated Pi electron system, while other, adjoining rings can be cycloalkyls, cycloalkenyls, cycloalkynyls, aryls, and/or heterocycles. The aryl group may be optionally substituted, as described herein. “Carbocyclic aryl groups” refer to aryl groups wherein the ring atoms on the aromatic ring are carbon atoms. Carbocyclic aryl groups include monocyclic carbocyclic aryl groups and polycyclic or fused compounds (e.g., two or more adjacent ring atoms are common to two adjoining rings) such as naphthyl group. Non-limiting examples of aryl groups include phenyl, naphthyl, tetrahydronaphthyl, indanyl, indenyl and the like.

The terms “heteroaryl” refers to aryl groups comprising at least one heteroatom as a ring atom, such as a heterocycle. Non-limiting examples of heteroaryl groups include pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like.

It will also be appreciated that aryl and heteroaryl moieties, as defined herein, may be attached via an aliphatic, alicyclic, heteroaliphatic, heteroalicyclic, alkyl or heteroalkyl moiety and thus also include -(aliphatic)aryl, -(heteroaliphatic)aryl, -(aliphatic)heteroaryl, -(heteroaliphatic)heteroaryl, -(alkyl)aryl, -(heteroalkyl)aryl, -(heteroalkyl)aryl, and -(heteroalkyl)-heteroaryl moieties. Thus, as used herein, the phrases “aryl or heteroaryl” and “aryl, heteroaryl, (aliphatic)aryl, -(heteroaliphatic)aryl, -(aliphatic)heteroaryl, -(heteroaliphatic)heteroaryl, -(alkyl)aryl, -(heteroalkyl)aryl, -(heteroalkyl)aryl, and -(heteroalkyl)heteroaryl” are interchangeable.

Any of the above groups may be optionally substituted. As used herein, the term “substituted” is contemplated to include all permissible substituents of organic compounds, “permissible” being in the context of the chemical rules of valence known to those of ordinary skill in the art. It will be understood that “substituted” also includes that the substitution results in a stable compound, e.g., which does not spontaneously undergo transformation such as by rearrangement, cyclization, elimination, etc. In some cases, “substituted” may generally refer to replacement of a hydrogen with a substituent as described herein. However, “substituted,” as used herein, does not encompass replacement and/or alteration of a key functional group by which a molecule is identified, e.g., such that the “substituted” functional group becomes, through substitution, a different functional group. For example, a “substituted phenyl group” must still comprise the phenyl moiety and cannot be modified by substitution, in this definition, to become, e.g., a pyridine ring. In a broad aspect, the permissible substituents include acyclic and cyclic, branched and unbranched, carbocyclic and heterocyclic, aromatic and nonaromatic substituents of organic compounds. Illustrative substituents include, for example, those described herein. The permissible substituents can be one or more and the same or different for appropriate organic compounds. For purposes of this invention, the heteroatoms such as nitrogen may have hydrogen substituents and/or any permissible substituents of organic compounds described herein which satisfy the valencies of the heteroatoms.

Examples of substituents include, but are not limited to, aliphatic, alicyclic, heteroaliphatic, heteroalicyclic, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, heteroalkylthio, heteroarylthio, sulfonyl, sulfonamido, ketone, aldehyde, ester, heterocyclyl, aromatic or heteroaromatic moieties, —CF₃, —CN, aryl, aryloxy, perhaloalkoxy, aralkoxy, heteroaryl, heteroaryloxy, heteroarylalkyl, heteroaralkoxy, azido, amino, halide, alkylthio, oxo, acylalkyl, carboxy esters, -carboxamido, acyloxy, aminoalkyl, alkylaminoaryl, alkylaryl, alkylaminoalkyl, alkoxyaryl, arylamino, aralkylamino, alkylsulfonyl, -carboxamidoalkylaryl, -carboxamidoaryl, hydroxyalkyl, haloalkyl, alkylaminoalkylcarboxy-, aminocarboxamidoalkyl-, cyano, alkoxyalkyl, perhaloalkyl, arylalkyloxyalkyl, (e.g., SO₄(R′)₂), a phosphate (e.g., PO₄(R′)₃), a silane (e.g., Si(R′)₄), a urethane (e.g., R′O(CO)NHR), and the like. Additionally, the substituents may be selected from F, Cl, Br, I, —OH, —NO₂, —CN, —NCO, —CF₃, —CH₂CF₃, —CHCl₂, —CH₂ORₓ, —CH₂CH₂ORₓ, —CH₂N(Rₓ)₂, —CH₂SO₂CH₃, —C(O)Rₓ, —CO₂(Rₓ), —CON(Rₓ)₂, —OC(O)Rₓ, —C(O)OC(O)Rₓ, OCO₂Rₓ, —OCON(Rₓ)₂, —N(Rₓ)₂, —S(O)₂Rₓ, —OCO₂Rₓ, —NRₓ(CO)Rₓ, —NRₓ(CO)N(Rₓ)₂, wherein each occurrence of Rₓ independently includes, but is not limited to, H, aliphatic, alicyclic, heteroaliphatic, heteroalicyclic, aryl, heteroaryl, alkylaryl, or alkylheteroaryl, wherein any of the aliphatic, alicyclic, heteroaliphatic, heteroalicyclic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. two or more atoms in common, wherein at least one ring comprises an oxygen atom.

The terms “carboxyl group,” “carbonyl group,” and “acyl group” are recognized in the art and can include such moieties as can be represented by the general formula:

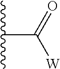

wherein W is H, OH, O-alkyl, O-alkenyl, or a salt thereof. Where W is O-alkyl, the formula represents an “ester.” Where W is OH, the formula represents a “carboxylic acid.” The term “carboxylate” refers to an anionic carboxyl group. In general, where the oxygen atom of the above formula is replaced by sulfur, the formula represents a “thiolcarbonyl” group. Where W is a S-alkyl, the formula represents a “thiolester.” Where W is SH, the formula represents a “thiolcarboxylic acid.” On the other hand, where W is alkyl, the above formula represents a “ketone” group. Where W is hydrogen, the above formula represents an “aldehyde” group.

As used herein, the term “halogen” or “halide” designates —F, —Cl, —Br, or —I.

The term “alkoxy” refers to the group, —O-alkyl.

The term “aryloxy” refers to the group, —O-aryl.

The term “acyloxy” refers to the group, —O-acyl.

The term “arylalkyl,” as used herein, refers to an alkyl group substituted with an aryl group.

The terms “amine” and “amino” are art-recognized and refer to both unsubstituted and substituted amines, e.g., a moiety that can be represented by the general formula: N(R′)(R″)(R″′) wherein R′, R″, and R″′ each independently represent a group permitted by the rules of valence.

“Alkylthio” as used herein alone or as part of another group, refers to an alkyl group, as defined herein, appended to the parent molecular moiety through a thio moiety, as defined herein. Representative examples of alkylthio include, but are not limited to, methylthio, ethylthio, tert-butylthio, hexylthio, and the like.

“Arylalkyl” as used herein alone or as part of another group, refers to an aryl group, as defined herein, appended to the parent molecular moiety through an alkyl group, as defined herein. Representative examples of arylalkyl include, but are not limited to, benzyl, 2-phenylethyl, 3-phenylpropyl, 2-naphth-2-ylethyl, and the like.

“Alkylamino” as used herein alone or as part of another group means the radical —NHR, where R is an alkyl group.

“Arylalkylamino” as used herein alone or as part of another group means the radical —NHR, where R is an arylalkyl group.

“Ester” as used herein alone or as part of another group refers to a —C(O)OR radical, where R is any suitable substituent such as alkyl, cycloalkyl, alkenyl, alkynyl or aryl.

The following examples are intended to illustrate certain embodiments of the present invention, but do not exemplify the full scope of the invention.

### Example 1

This example describes the synthesis and use cobalt hangman corrole bearing β-octafluoro and meso-pentafluorophenyl substituents as active water-splitting catalysts. When immobilized in Nafion films, the turnover frequencies for the 4 e⁻/4H⁺ process at the single cobalt center of the hangman platform approached 1 s⁻¹. The pH dependence of the water splitting reaction suggested a proton-coupled electron transfer (PCET) catalytic mechanism.

An attractive approach to meeting the energy demands of a growing global population is to capture solar energy and store it in the form of chemical fuels. A prevailing solar-to-fuels process is the splitting of water to produce hydrogen, which may be used directly or combined with carbon dioxide to produce a more conventional fuel.

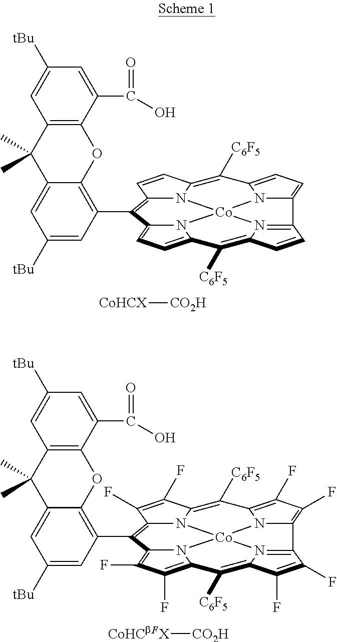

The water splitting process is a 4 e⁻/4H⁺ process, thus providing an imperative for the discovery of new catalysts that bridge the 1 e⁻/1H⁺ capture process of most light harvesting systems including semiconductors, to the 4 e⁻/4H⁺ process of water splitting.

Co(II) hangman porphyrins can promote the 4 e⁻/4H⁺ reduction of oxygen to water. The oxygen reduction reaction (ORR) is the reverse of the oxygen evolving reaction (OER). Moreover, the oxygen atoms from two water molecules may be situated within the hangman cleft; one water is in the primary coordination sphere of the metal whereas another is held in the secondary coordination sphere via hydrogen bonding to the hanging group. This example describes the synthesis of β-octafluoro hangman corrole (Scheme 1), CoH^{βF}CX—CO₂H, and it's use as an OER catalyst.

Hangman corroles may be synthesized concisely from easily available starting materials, in two steps, in good yields and with abbreviated reaction times. In order to boost the oxidizing power of the corrole subunit, the macrocycle was modified with electron-withdrawing groups. Introduction of ancillary fluorinated phenyl groups onto the 5 and 15 meso-positions of the framework increased the oxidizing power of the macrocycle by more than 0.4 V and an additional 0.5-0.6 V was gained upon fluorination of the β-pyrrolic positions of the macrocycle. The β-octafluoro hangman corrole was synthesized in 23% overall yield. ¹⁹F NMR established that the trans-A₂B isomer was obtained. The relatively high yield in part is due to the use of microwave irradiation, which efficiently drives metal insertion and deprotection of the hanging methyl ester group. Syntheses and characterization details of the compounds are provided in Example 2.

Samples for electrocatalysis were prepared by dissolving the corrole in a 10:2:1 mixture of THF:ethanol:Nafion solution, to give a final concentration of 0.5% Nafion and 1 mM catalyst. A 30 μL drop of solution was then deposited onto an FTO coated glass slide and allowed to slowly evaporate. The resulting film contained 30 nmol of catalyst per cm².

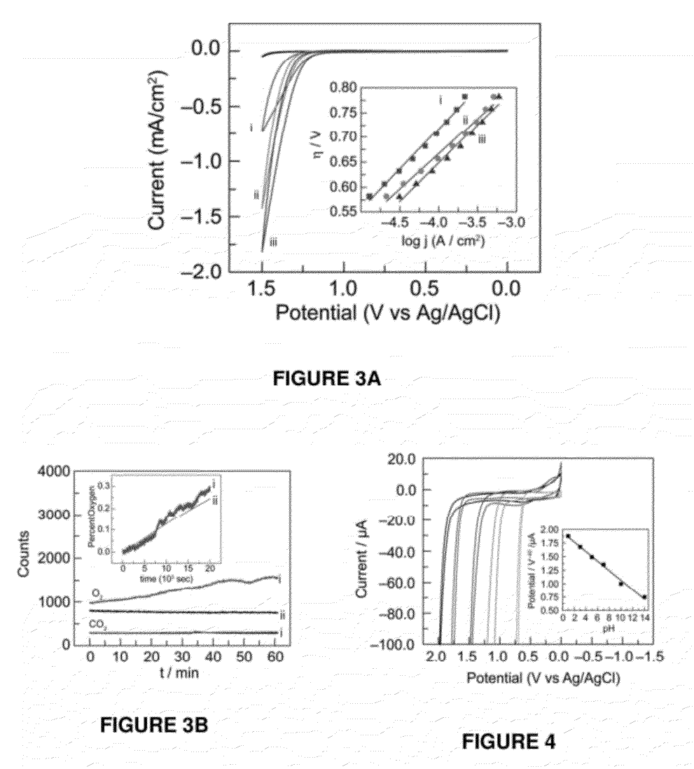

A cyclic voltammogram (CV) of CoH^{βF}CX—CO₂H in CH₂Cl₂, was obtained. CoH^{βF}CX—CO₂H exhibited a reduction wave at 0.33 V and an oxidation event at 0.87 V vs ferrocene. Addition of water to a DMF solution of the corrole revealed a catalytic peak positive of the reversible oxidation process prompting the examination of the electrochemistry aqueous solutions (0.1 M phosphate buffer, pH=7). FIG. 3a shows that CoH^{βF}CX—CO₂H exhibited greater current and earlier onset of catalytic current as compared to its β-nonfluorinated congener, and both hangman compounds exhibited greater current than the cobalt corrole possessing meso-C₆F₅ groups but lacking the hanging group (Co(C₆F₅)₃). The onset of the catalytic current (1.25 V vs Ag/AgCl) occurred ca. 0.6 V beyond the thermodynamic potential for water oxidation at pH=7 (0.62 V vs Ag/AgCl); this value is nearly 100 mV higher than recently reported molecular cobalt catalysts. The inset in FIG. 3a shows the Tafel plots of the three corrole compounds. A slope of ca. 120 mV/decade indicates that an electron transfer step may be the rate-determining step. The curvature of the Tafel plot indicates a potentially more complex mechanism, however, this curvature may be due the mediation of electron and proton transfer by the Nafion film and not to mechanistic details associated with the intrinsic activity of the catalyst. The “Co^{IV}” hangman corroles were isolable species. Spectroscopic properties and DFT calculations of cobalt corrole axially ligated by chloride are consistent with the “Co^{IV}” species, which may be better described as a Co^{III} site strongly antiferromagnetically coupled to the S=½ of the monoradical dianion corrole, [Co^{III}Cl-corrole^{+•}]. β-fluorination may make the corrole more difficult to oxidize and therefore, the exact oxidation state of the metal and corrole remains unclear. Regardless of the exact nature of the pre-catalytic state, water oxidation occurred more positive of the first oxidation event of Co^{III}HC^{βF}X—CO₂H to produce formally Co^{III}H^{+•}C^{βF}X—CO₂H or Co^{IV}HC^{βF}X—CO₂H. These results indicate that the active catalytic species may be Co^{IV}H^{+•}C^{βF}X—CO₂H.

The Faradaic efficiency of the CoHC^{βF}X—CO₂H catalyst was measured by using a fluorescence-based O₂ sensor. Electrolysis was performed in aqueous solutions (0.1 M phosphate buffer, pH=7) in a gas-tight electrochemical cell under an N₂ atmosphere with the sensor placed in the headspace. After initiating electrolysis at 1.4 V, the percentage of O₂ detected in the headspace rose in accord with what was predicted by assuming that all of the current was caused by 4 e⁻ oxidation of water to produce O₂ (FIG. 3b inset). Thus substantially only O₂ is produced during catalytic turnover by CoHC^{βF}X—CO₂H.

In FIG. 3a: Cyclic voltammograms of FTO background and Nafion films containing 30 nmol/cm² of Co(C₆F₅) (i), CoHCX—CO₂H (ii) and CoH^{βF}CX—CO₂H (iii) deposited on FTO glass in 0.1 M KPi buffer, pH 7. Scan rate, 0.1 V/s. Inset: Tafel plots of the same electrodes. In FIG. 3b: Mass spectrometric detection of O₂ and CO₂ during electrolysis of a bare electrode (ii) and CoH^{βF}CX—CO₂H (i) at 1.4 V vs Ag/AgCl. Inset: O₂ production measured by fluorescent sensor (i) and the theoretical amount of O₂ produced (i) assuming a Faradic efficiency of 100%.

The gaseous products evolved during electrolysis at constant potential (1.4 V vs Ag/AgCl, pH=7) were measured by mass spectrometry. Substantially only O₂ was produced during catalytic turnover. If H₂O₂ was formed, then it may rapidly dismutate because hangman complexes are known to be very active catalysts for this reaction. By this measurement, CoH^{βF}CX—CO₂H was also observed to be the most efficient catalyst of the series as nearly twice as much oxygen was detected as compared to CoHCX—CO₂H. With OER established, the turnover frequency (TOF) may be calculated by measurement of the current density for the 4 e⁻/4H⁺ OER process. At 1.4 V vs Ag/AgCl, the TOFs per Co atom for CoHCX—CO₂H and CoHC^{βF}X—CO₂H are 0.55 s⁻¹ and 0.81 s⁻¹, respectively.

These numbers compare favorably with regard to other cobalt-based water oxidation catalysts. Importantly, during the course of the electrolysis, no CO₂, was observed which may result if the corrole framework were to be oxidized. As further testament to the catalyst stability, the CoHC^{βF}X—CO₂H electrode was immersed in THF upon the completion of electrolysis and the solution was concentrated. UV-vis, LD-MS MALDI-TOF and high resolution ESI-MS indicate the presence of primarily the cobalt hangman corrole. Finally the pH dependence of the OER reaction was well-behaved. FIG. 4 shows that the overpotential for OER decreased with increasing pH. A plot of the potential as measured at constant current (40 μA) versus pH shows a monotonic decrease from pH=14 to pH=1 (FIG. 4, inset). This indicates that decomposition of the catalyst to cobalt oxide is unlikely, as such species are unstable in very acidic solutions. Finally, the onset potentials for the three catalysts are unique and the overpotential for OER is also inconsistent with cobalt oxide type species.

In FIG. 4: pH dependence of cyclic voltammograms at 1, 3, 7, 10 and 14 from left to right. Scan rate 100 mV/s. Inset: Potential (measured at 40 μA) vs pH with a slope of 88 mV/pH unit.

In summary, hangman cobalt corroles are OER catalysts with β-octafluoro Co^{III} xanthene hangman corrole bearing, 5,15-bis(pentafluorophenyl) substituents were effective OER catalysts. They were more active than its non-hangman analogues and activity was augmented by the fluorination of the corrole macrocycle. The catalysts were stable under operating conditions and evolved only oxygen as the OER product at modest overpotential. The ability of the hanging group to pre-organize water within the hangman cleft appears to be beneficial for the 0-0 bond forming reaction of OER.

### Example 2

This example provides supporting information for Example 1. General Methods. ¹H NMR spectra (500 MHz) were recorded on samples in CDCl₃ at room temperature unless noted otherwise. Silica gel (60 μm average particle size) was used for column chromatography. Compounds Co(C₆F₅)₃, HCX—CO₂H, CoC(C₆F₅)O₃, and CoHCX—CO₂H were prepared as described in the literature. The synthesis and characterization of CoH^{βF}CX—CO₂H is described below.

THF (anhydrous), methanol (anhydrous), CH₂Cl₂ (anhydrous), CHCl₃ and all other chemicals were reagent grade and were used as received. 3,4-difluoropyrrole was purchased from Frontier Scientific Inc. LD-MS data were collected in the absence of matrix. UV-vis spectra were recorded at room temperature in quartz cuvettes in anhydrous THF on a Varian Cary 5000 UV-vis-NIR spectrophotometer. Steady state emission spectra were recorded on an automated Photon Technology International (PTI) QM 4 fluorimeter equipped with a 150-W Xe arc lamp and a Hamamatsu R928 photomultiplier tube. Excitation light was wavelength selected with glass filters. Solution samples were prepared under ambient atmosphere in anhydrous THF and contained in screw-cap quartz fluorescence cells.

The microwave-assisted metallation of free-base hangman porphyrins was performed inside the cavity of a CEM Discover microwave synthesis system equipped with infrared, pressure, and temperature sensors for monitoring the synthesis. The reaction vessels were 10 mL crimp-sealed thick-wall glass tubes. The contents of each vessel were subject to magnetic stirring.

Synthesis. The syntheses of β-octafluoro hangman corroles are shown in Scheme 2:

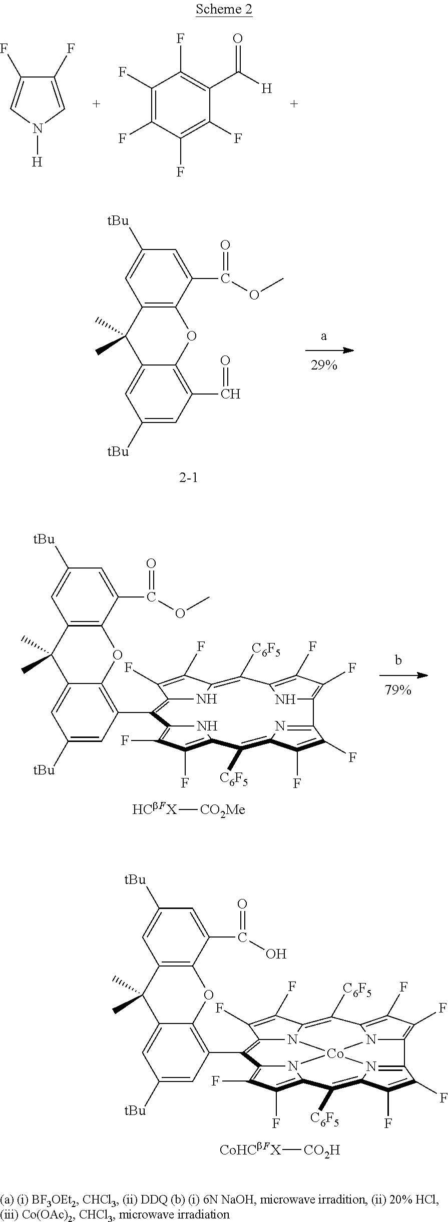

2,3,7,8,12,13,17,18-Octafluoro-10-(4-(5-Methoxycarbonyl-2,7-di-tert-butyl-9,9-dimethyl-xanthene))-5,15-bis(pentafluorophenyl)corrole (H^{βF}CX—CO₂Me). By modifying the statistical Lindsey synthesis (Lindsey, J. S.; Wagner, R. W. J. Org. Chem. 1989, 54, 828.) chloroform (425 mL) was placed in an oven dried round bottom flask (1000 mL) and purged with high flow of argon for 1 h. 3,4-difluoropyrrole (0.412 g, 4.00 mmol) was added. The reaction flask was covered with aluminium foil and purged with argon for 45 min. A sample of pentafluorobenzaldehyde (0.460 mL, 3.75 mmol), and xanthene backbone (2-1) (0.100 g, 0.250 mmol) were added. The resulting mixture was purged with argon in dark for additional 45 min. [Note 1: Longer purging time results mainly formation of β-octafluoro 5,10,15,20-tetrakis(pentafluorophenyl)porphyrin]. A sample of BF₃.OEt₂ (0.168 mL, 1.32 mmol) was added drop wise via syringe. The reaction mixture was stirred under argon in dark for 1 h. A sample of DDQ (0.453 g, 2.00 mmol) was added in once and the resulting mixture was stirred for 1 h. The resulting crude reaction mixture was concentrated to dryness and chromatographed (hexanes:CH₂Cl₂. (10:1)→hexanes:CH₂Cl₂ (5:2)) affording a purple solid (83 mg, 29%). ¹H NMR (500 MHz, CDCl₃) δ/ppm: 1.24 (s, 9H), 1.52 (s, 9H), 1.79 (s, 3H), 1.85 (s, 6H), 7.30 (d, J=2.5 Hz, 1H), 7.64 (d, J=2.5 Hz, 1H), 7.83 (d, J=2.5 Hz, 1H), 7.95 (d, J=2.5 Hz, 1H), pyrrolic protons were not observed at room temperature. ¹⁹F NMR (CFCDCl₃ was used as an external standard for calibration), −140.73, −147.87, −151.66, −153.69, −157.88, −162.21. HR(ESI)-MS (M−H⁺) (M=C₅₆H₃₄F₁₈N₄O₃): Calcd for m/z=1151.2271, obsd 1151.2221. LD-MS obsd 1153.73. λ_{max,abs}/nm (CH₂Cl₂)=403, 498, 536, 575, 626. λ_{max,em}(403 exc)/nm=654. Anal. Calcd for C₅₆H₃₄F₁₈N₄O₃: C, 58.34; H, 2.97; N, 4.86; Found: C, 58.92; H, 2.07; N, 4.25.

2,3,7,8,12,13,17,18-Octafluoro-10-(4-(5-Hydroxycarbonyl-2,7-di-tert-butyl-9,9-dimethyl-xanthene))-5,15-bis(pentafluorophenyl)corrole (HC^{βF}X1-CO₂H). A solution of HC^{βF}X1-CO₂Me (50.0 mg, 0.0434 mmol) in THF (2 mL) was treated with 6 N NaOH (2 mL). The reaction mixture was subjected to microwave irradiation for 16 h. The reaction mixture was diluted with CH₂Cl₂ (100 mL), washed (water, brine) and dried with Na₂SO₄. The crude product was concentrated approximately to 20 mL and treated with 20% HCl (50 mL). The biphasic reaction mixture was stirred overnight. The organic phase was separated with CH₂Cl₂, washed (water, and brine), and dried with Na₂SO₄. The crude product was chromatographed (silica, CH₂Cl₂) to afford a purple solid (42 mg, 86%). ¹H NMR (500 MHz, CDCl₃) δ/ppm: 1.25 (s, 9H), 1.58 (s, 9H), 1.95 (s, 6H), 7.62-7.66 (br.s, 1H), 7.46-7.49 (br. s, 1H), 7.86-7.88 (m, 1H), 7.89-7.92 (br. s, 1H), pyrrolic (3H) and carboxylic acid protons were not observed at room temperature. HR(ESI)-MS (M−H⁺) (M=C₅₅H₃₂F₁₈N₄O₃): Calcd for m/z=1137.2114, obsd 1137.2093. LD-MS obsd 1139.82. λ_{max,abs}/nm (CH₂Cl₂)=400, 497, 533, 625. λ_{max,em}(400 exc)/nm=646.

2,3,7,8,12,13,17,18-Octafluoro-10-(4-(5-Hydroxycarbonyl-2,7-di-tert-butyl-9,9-dimethyl-xanthene))-5,15-bis(pentafluorophenyl) cobalt corrole (CoHC^{βF}X—CO₂H). A microwave glass tube (10 mL) containing a magnetic stir bar was charged with CHCl₃ (2 mL) and HC^{βX}—CO₂H (22.0 mg, 0.0193 mmol). The solution was stirred at room temperature for 10 min to obtain a homogenous mixture. Co(OAc)₂ (17.0 mg, 0.0965 mmol, 5 mol equiv. versus corresponding HC^{βF}X1-CO₂H). The resulting mixture was stirred at room temperature for 10 min after which the reaction vessel was sealed with a septum and subjected to microwave irradiation at 60° C. The protocol was as follows: (1) heat the reaction vessel from room temperature to 60° C., (2) hold at 60° C. and irradiate for 30 min (temperature overshoots of 65-70° C. were permitted; temperature was re-established at 60° C. by using open flow valve option), (3) allow the reaction mixture to cool to room temperature, (4) check the reaction mixture by silica TLC analysis, (5) repeat steps 1-4 until all of the starting material was consumed (2 h). Upon complete reaction, the crude reaction mixture was checked with MALDI-TOF. A sample of triethylamine (5 mol equiv to metal salt) was added to the solution, which was washed with water and brine, dried over Na₂SO₄, and concentrated to dryness. The resulting crude product was chromatographed (silica, CHCl₃) to afford the purple solid. (21, 92%). ¹H NMR (500 MHz, CDCl₃) δ/ppm: 1.26 (s, 9H), 1.51 (s, 9H), 1.95 (s, 6H), 7.59 (d, J=2.5 Hz, 1H), 7.76 (d, J=2.5 Hz, 1H), 7.88 (dd, J=2.5 Hz, 2H), 8.54-8.84 (br. s, 1H). HR(ESI)-MS (M−H⁺) (M=C₅₅H₂₉CoF₁₈N₄O₃): Calcd for m/z=1193.1201, obsd 1193.1177. LD-MS obsd 1194.40. A_{max,abs}/nm (CH₂Cl₂)=408, 488, 529, 568. Anal. Calcd for C₅₅H₂₉CoF₁₈N₄O₃: C, 55.29; H, 2.45; N, 4.69; Found: C, 55.93; H, 3.07; N, 5.18

Electrochemistry. Electrocatalytic activity was measured in a 3-electrode H-cell in 0.1 M phosphate buffer. The counter electrode was Ni foam and the reference was Ag/AgCl electrode from BAS instruments. For oxygen detection, the solution was bubbled with N₂ for 30 min and then the electrochemical cell was evacuated several times and allowed to equilibrate overnight with the He carrier gas. All potentials are reported versus Ag/AgCl. pH dependent measurements were made in 0.5 M H₂SO₄, pH 1; 0.5 M KOH, pH 14; 0.1 M phosphate buffer adjusted to pH 3, 5, 10 with either 0.5 M H₂SO₄ or 0.5 M KOH.

The cyclic voltammogram (CV) of CoH^{βF}CX—CO₂H was recorded in CH₃CN solutions containing 0.1 M NBu₄PF₆ (tetrabutylammonium hexafluorophosphate) and the corrole compound. A three compartment cell was employed possessing a 0.07 cm² glassy carbon button electrode as the working electrode, Pt wire as the auxiliary electrode, and Ag/AgCl as a reference electrode. CVs were collected with scan rates of 10-100 mV/s with iR compensation.

The absorption and emission spectra of HC^{βF}X—CO₂Me in CH₂Cl₂ at room temperature showed peaks as 403 (max) 498, 536, 575, and 626 nm (absorption) and 654 (max) mn (emission), respectively.

The absorption and emission spectra of HC^{βF}X—CO₂H in CH₂Cl₂ at room temperature showed peaks at 400 (max), 497, 533, and 625 (absorption), and 646 (max) nm (emission), respectively.

The absorption spectrum of CoHC^{βF}X—CO₂H in CH₂Cl:EtOH (3:1) at room temperature showed peaks at 408 (max), 288, 529, and 568 nm.

Cyclic voltammograms (CV) of CoHC^{βF}X—CO₂H (top) and Co(C₆F₅) and CoHCX—CO₂H were recorded in CH₂Cl₂ solutions containing 0.1 M NBu₄PF₆ (tetrabutylammonium hexafluorophosphate). A three compartment cell was employed possessing a 0.07 cm² glassy carbon button electrode as the working electrode. Pt wire was used as the auxiliary electrode, and Ag/AgCl as a reference electrode. CVs were collected with scan rates of 50 mV/s with iR compensation.

Cyclic voltammogram (CV) of CoHC^{βF}X—CO₂H in DMF solutions containing 0.1 M NBu₄PF₆ (tetrabutylammonium hexafluorophosphate) were recorded. Addition of alliquots of water: 100 μL (i), 200 μL (ii) and 300 μL (iii). A three compartment cell was employed possessing a 0.07 cm² glassy carbon electrode as the working electrode. Pt wire was used as the auxiliary electrode, and Ag/AgCl as a reference electrode. CVs were collected with scan rates of 100 mV/s.

The absorption spectrum of CoHC^{βF}X—CO₂H in CH₂Cl:EtOH (3:1) at room temperature before and after 30 min electrolysis at 1.4 V vs Ag/AgCl showed peaks at 408 (max), 488, 529, and 568 nm (before), and 408 (max), 529, and 568 nm (after), respectively.

The LD-MS of CoHC^{βF}X—CO₂H (exact mass: 1194.1284; mol. wt.: 1194.7482) after 30 min electrolysis at 1.4 V vs Ag/AgCl showed peaks at 1140.28 and 1194.32.

The ESI-MS of CoHC^{βF}X—CO₂H after 30 min electrolysis at 1.4 V vs Ag/AgCl (recovered sample) showed peaks at 691.9812, 734.0102, 805.9915, 982.9956, 1033.9839, 1047.9074, 1194.1237 ([M−H]⁻), 1210.1446, 1240.1400, and 1324.1282.

The ESI-MS of CoHC^{βF}X—CO₂H after 30 min electrolysis at 1.4 V vs Ag/AgCl showed peaks at 1193.1177, 1194.1237, 1195.1317, and 1196.1335.

Cyclic voltammogram (CV) of CoHC^{βF}X—CO₂H in 0.1 M phosphate buffer pH 7 before (i) and after (ii) 30 min electrolysis at 1.4 V vs Ag/AgCl were recorded.

Mass spectrometric detection of O₂ and CO₂ during electrolysis at 1.4 V vs Ag/AgCl: Co(C₆F₅) (i), CoHCX—CO₂H (ii) and CoH^{βF}CX—CO₂H (iii) was recorded, as well as the charge passed for each electrode during the electrolysis.

Cyclic voltammograms (CV) of CoHCX—CO₂H at pH 1 (vi), pH 3 (i), pH 5 (iii), and pH 7 (ii) were recorded, as well as potential at constant current (−40 μA) versus pH.

### Example 3

This example describes a cobalt(II) hangman porphyrin with a xanthene backbone and a carboxylic acid hanging group which catalyzes the electrochemical production of hydrogen from benzoic and tosic acid in acetonitrile solutions. A Co^{II}H species was likely involved in the generation of H₂ from weak acids. In a stronger acid, a Co^{III}H species was observed electrochemically, but may need to be further reduced to Co^{II}H before H₂ generation occurs. Overpotentials for H₂ generation were lowered as a result of the hangman effect.

Hydrogen generation from carbon neutral sources is an important element of a multi-faceted strategy to meet growing global energy demands. Accordingly, renewed interest in H₂ catalyst discovery has led to the creation of a variety of complexes that electrocatalyze H⁺ reduction. A particularly fascinating design element of emergent catalysts is the incorporation of a proton relay from a pendant acid-base group proximate to the metal center where H₂ production occurs. These catalysts are akin to the active sites of hydrogenases, which feature pendant bases positioned near the metal centers that are postulated to play a role in enzyme catalysis. The benefits of a pendant proton relay are consistent with the early proposal of H₂ generation via the pathway shown in Scheme 3A: reduction of a Co^{II} center to Co^{I} followed by H⁺ attack to yield a hydridic Co^{III}H species that yields H₂ upon protonolysis or bimetallic reaction. However, this mechanism has been recently re-considered in view of the contention that Co^{III}H centers may not be sufficiently basic to drive protonolysis and it has been suggested that more reduced cobalt species must be attained before protonolysis can occur (Scheme 3B). The inability to control proton stoichiometry in most catalytic cycles has made it difficult to distinguish mechanisms and thus discern which intermediate is involved in catalysis. On this count, the utility of hangman active sites may provide insight into the mechanism of H₂ evolution by stoichiometric generation of a key intermediate as a result of the hangman effect. In the hangman construction, an acid-base functionality is positioned from a xanthene or furan spacer over the face of a redox-active macrocycle such as porphyrin, salen or corrole. The acid-base hanging group may permit the facile transfer of a single proton to and/or from a substrate bound to the metal macrocycle. With the ability to control proton stoichiometry from the hanging group, studies were completed to examine H₂ generation at CoHPX—CO₂H (1-Co) shown in Scheme 4. Comparison of the electrochemistry of 1-Co to a macrocyclic analog in which the hanging group has been removed CoHPX—Br (2-Co, Scheme 4) establishes the hangman effect (via a

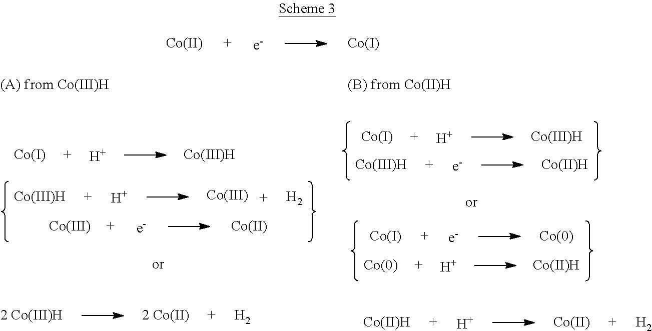

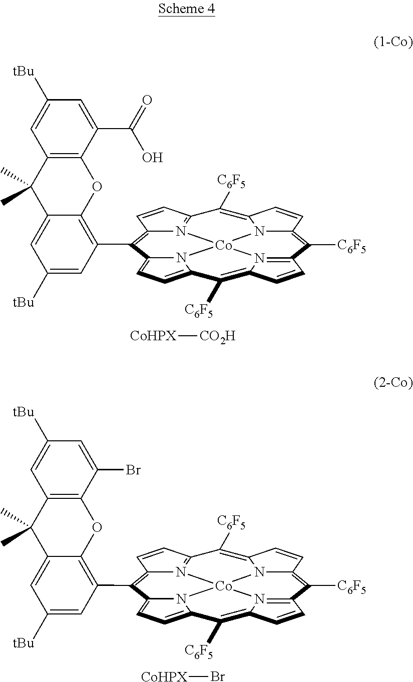

reduced overpotential) and that the Co center produces H₂ only beyond reduction potentials exceeding the Co^{I} oxidation state. The results are described in this example are consistent with the generation of Co^{II}H as a key intermediate in H₂ electrocatalysis at the hangman cobalt porphyrin active sites.

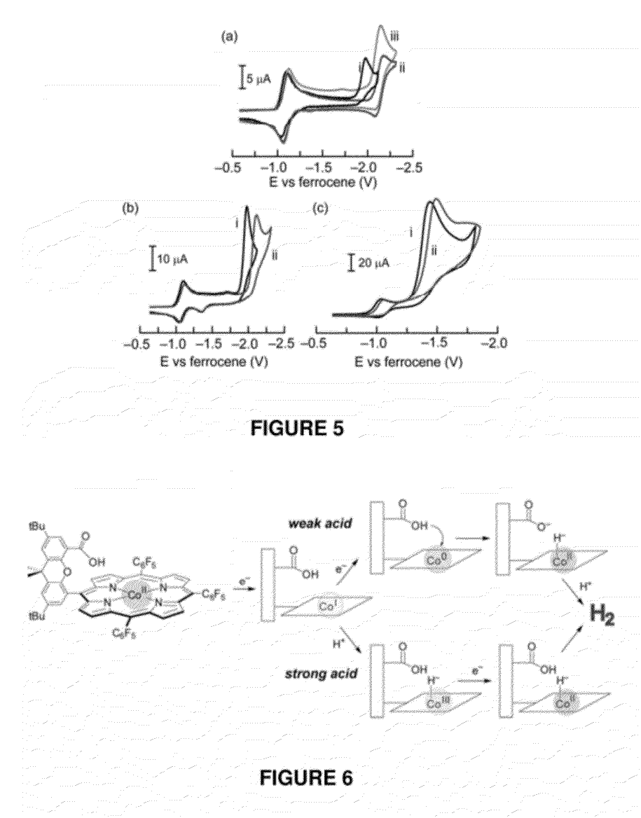

Hangman porphyrins can be obtained in appreciable quantities, in short synthesis times, and in high yields. 1-Co and 2-Co were synthesized following these methods. As shown in FIG. 5a, 1-Co and 2-Co exhibit reversible waves for the Co^{II/I} couple at almost the same potentials (−1.08 V vs the ferrocene/ferrocenium couple for 1-Co and −1.10 V for 2-Co). The cyclic voltammogram (CV) of 2-Zn shows redox waves at −1.52 and −1.92 V which indicates that each electrochemical feature of 1-Co and 2-Co has significant cobalt character. For simplicity, the reduction potentials are formally ascribed to Co, though there may be significant electron density on the porphyrin ring at very reducing potentials.

Whereas 2-Co shows a reversible wave for Co^{I/0} at −2.14 V, interestingly, 1-Co produced an irreversible wave for the reduction of Co^{I} and the wave was positively shifted by −200 mV. The major structural difference between 1-Co and 2-Co is the hanging carboxylic acid group and accordingly the irreversible process of 1-Co may be due to the hangman effect where the reduction of Co^{I} to Co⁰ is followed by immediate proton transfer from the hanging group to produce Co^{II}H. The second wave in the CV of 2-Co was irreversible upon the addition of external benzoic acid. At 1 equiv of benzoic acid, the wave began to exhibit irreversibility, also indicating protonation of the Co⁰ species. Irreversibility of the wave was observed only upon addition of >1 equiv of benzoic acid; this observation may be consistent with the hangman effect in 1-Co.

In FIG. 5: (a) CVs of 0.5 mM of 1-Co (i), 2-Co (ii), and 2-Co in the presence of 0.5 mM benzoic acid (iii). (b) CV of 0.5 mM of 1-Co in the presence of 2.5 mM benzoic acid (i) and 0.5 mM of 2-Co in the presence of 3.0 mM benzoic acid (ii). (c) CV of 0.8 mM of 1-Co (i) and 2-Co (ii) in the presence of 10 mM tosic acid. Scan rate, 100 mV/s; 0.1 M NBu₄PF₆ in acetonitrile. Glassy carbon working electrode, Ag/AgNO₃ reference electrode and Pt wire counter electrode.

In the presence of excess benzoic acid (pKₐ=20.7 in acetonitrile), 1-Co and 2-Co exhibited catalytic cathodic waves (FIG. 5b). The overpotential for catalysis was large (˜800 mV). The CV features of the electrocatalysis uncover essential mechanistic details of HER at cobalt macrocycles. The Co^{II/I} reduction feature was not affected much by the presence of acid but the second reduction wave exhibited pronounced catalytic activity. These results indicate, in this embodiment, that benzoic acid is too weak an acid to protonate the Co^{I} center, and hence catalytic H₂ production was observed only upon further reduction to Co⁰. The overpotential for proton reduction of 1-Co is −120 mV lower potential than that of 2-Co at 3 mM acid concentration. Moreover, the potential of the second reduction wave of 1-Co was the same in the presence and absence of acid. This is not the case for 2-Co; with increasing acid concentration the wave shifts to lower potential by 80 mV. These results are also indicative of the hangman effect since in 1-Co, proton transfer may not be rate-determining for catalysis (hence the insensitivity of the reduction wave to proton concentration) whereas in 2-Co, the proton transfer may be a determinant of the mechanism (hence the shift to lower potential with increasing acid). For either case, H₂ catalysis is initiated from the Co^{II}H.

Bulk electrolysis was performed in acetonitrile solutions of 0.4 mM 1-Co at −2.05 V and of 0.5 mM 2-Co at −2.20 V in the presence of 15 mM benzoic acid. The amount of H₂ gas produced during the electrolysis was determined by gas chromatography after 15 C of charges had passed. Faradaic efficiencies for H₂ production were ca. 80% and 85% for 1-Co and 2-Co, respectively; no other gaseous product was detected in the experimental condition. On the basis of TLC, mass spectra and UV-vis measurements, the decomposed product in bulk electrolysis in the presence of 2-Co does not correspond to a demetallated porphyrin or other porphyrin product.

In the presence of the stronger tosic acid (pKₐ=8.3 in acetonitrile), both 1-Co and 2-Co exhibited catalytic cathodic waves at ˜−1.5 V (FIG. 5c). The similarity of the CVs with regard to current and the onset of electrocatalysis suggests that the stronger acid might have overwhelmed the chemistry of the system and the hangman effect was obviated. As observed for benzoic acid, electrocatalysis for 1-Co and 2-Co occurred at potentials negative of the Co^{II/I} couple. However, there was one significant difference between the benzoic acid and tosic acid data; unlike the situation for benzoic acid, the Co^{II/I} wave became irreversible in the stronger tosic acid for both 1-Co and 2-Co (FIG. 5c). This indicates that Co^{I} may be protonated by the tosic acid. But the observation that catalysis occurs well past the Co^{II/I} reduction event indicates that a Co^{III}H species, when formed, may be further reduced to Co^{III}H for H₂ generation to occur. One determinant of the metal basicity is the presence of meso groups on macrocycle periphery. The electron withdrawing C₆F₅ groups may attenuate the metal center basicity and make the metal less reactive to protons.

In summary, the hangman porphyrin provides mechanistic insight into H⁺ reduction owing to the ability to control proton equivalency precisely via the hanging group. The irreversibility and positive shift of the reduction of Co^{I} in 1-Co together with a lowered overpotential for H₂ production are a result of the hangman effect. For the case of weak acids, H₂ was produced upon reduction to Co⁰ followed by protonation (middle bracket, Scheme 3B). For stronger acids, Co^{I} was first protonated and electron reduction follows it (top bracket, Scheme 3; Also see FIG. 6). Regardless of the strength of the acid, these results are consistent with H₂ production being mediated by Co^{II}H. Further reduction of the metal may allow for the effective protonation of the hydride to produce H₂.

### Example 4

This example provides supporting information for Example 3.

General Methods. ¹H NMR spectra (500 MHz) were recorded on samples in CDCl₃ at room temperature unless noted otherwise. Silica gel (60 μm average particle size) was used for column chromatography. 4-Formyl-5-bromo-2,7-di-tert-butyl-9,9-dimethylxanthene (3) 5-pentafluorophenyldipyrro-methane (4), 1,9-bis(pentafluorobenzoyl)-5-(pentafluorophenyl)dipyrromethane (5), 1,9-bis(pentafluorobenzoyl)-5-(pentafluorophenyl)dipyrromethane dicarbinol (5-OH), 5-(4-(5-hydroxycarbonyl-2,7-di-tert-butyl-9,9-dimethyl-xanthene))-10,15,20-tris(pentafluorophenyl)-porphyrinatocobalt(II) (1-Co) were prepared as described in the literature. THF (anhydrous), methanol (anhydrous) and CH₂Cl₂ (anhydrous) and all other chemicals were reagent grade and were used as received. LD-MS data was measured on porphyrins in the absence of matrix.

The microwave-assisted reactions were performed inside the cavity of a CEM Discover microwave synthesis system equipped with infrared, pressure and temperature sensors for monitoring the synthesis. The reaction vessels were 10 mL crimp-sealed thick-wall glass tubes. The contents of each vessel were stirred with a magnetic stirrer.

UV-vis spectra were recorded at room temperature in quartz cuvettes in anhydrous CH₂Cl₂ on a Varian Cary 5000 UV-vis-NIR spectrophotometer. Steady state emission spectra were recorded on an automated Photon Technology International (PTI) QM 4 fluorimeter equipped with a 150-W Xe arc lamp and a Hamamatsu R928 photomultiplier tube. Excitation light was wavelength selected with glass filters. Solution samples were prepared under ambient atmosphere in anhydrous CH₂Cl₂ and contained in screw-cap quartz fluorescence cells.

### Synthesis

### 5-(4-(5-bromo-2,7-di-tert-butyl-9,9-dimethylxanthene))dipyrromethane (6)

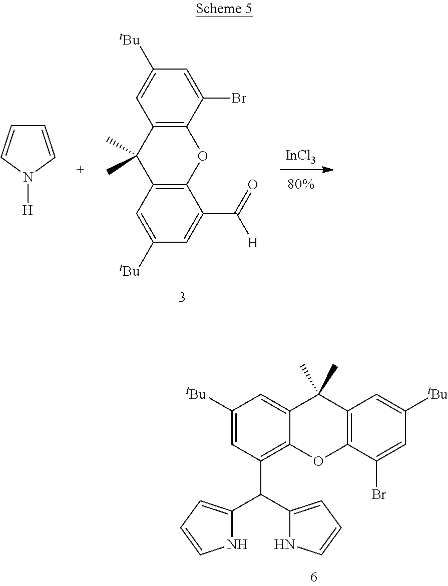

A mixture of 4-formyl-5-bromo-2,7-di-tert-butyl-9,9-dimethylxanthene 3, (1.00 g, 2.33 mmol) and pyrrole (15.6 mL, 233 mmol) in a 50-mL flask was degassed with a stream of argon for 10 min at room temperature. The mixture was heated to 75° C. to obtain a clear solution. InCl₃ (50.0 mg, 0.226 mmol) was then added, and the mixture was stirred at 75° C. for 2 h. A sample of NaOH (0.280 g, 7.00 mmol) was added and the mixture was stirred at 75° C. for 1.5 h. The mixture was filtered. The filtrate was concentrated and resulting crude product was chromatographed [silica, hexanes:CH₂Cl₂ (3:2)] to afford a light yellow foam solid (1.01 g, 80%). ¹H NMR (500 MHz, CDCl₃) δ/ppm: 1.27 (s, 9H), 1.32 (s, 9H), 1.63 (s, 6H), 6.02 (br. s, 2H), 6.14-6.16 (m, 3H), 6.69-6.70 (m, 2H), 7.18 (d, J=2.5 Hz, 1H), 7.28 (d, J=2.5 Hz, 1H), 7.35 (d, J=2.5 Hz, 1H), 7.43 (d, J=2.5 Hz, 1H), 8.24-8.44 (br. s, 2H). Anal. Calcd. for (M+H⁺), M=C₃₂H₃₇BrN₂O, Calcd. 545.2162. Found for HR(ESI)-MS: 545.2162. Anal. Calcd. for C₃₂H₃₇BrN₂O: C, 70.45; H, 6.84; N, 5.13. Found: C, 70.95; H, 6.90; N, 5.09.

### 5-(4-(5-Bromo-2,7-di-tert-butyl-9,9-dimethylxanthene))-10,15,20-tris(pentafluorophenyl)-porphyrin (HPX—Br, 2) (statistical synthesis, Scheme 6)

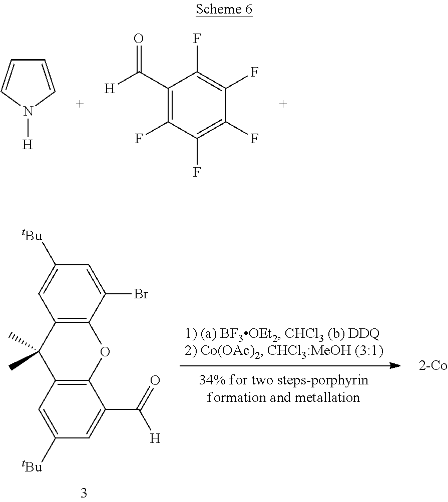

By following the statistical Lindsey porphyrin forming reaction, (e.g., see Lindsey, J. S.; Wagner, R. W. J. Org. Chem. 1989, 54, 828-836) CHCl₃ (425 mL) was placed in an oven dried round bottom flask (1000 mL) and purged with high flow of argon for 1 h. Pyrrole (0.275 mL, 4.00 mmol) was added via syringe to the reaction flask, which was covered with aluminum foil and the solution was purged with argon for 45 min. The pentafluorobenzaldehyde (0.735 g, 3.75 mmol) and 4-formyl-5-bromo-2,7-di-tert-butyl-9,9-dimethylxanthene 3, (0.107 g, 0.25 mmol) were then added to the round bottom. The resulting mixture was purged with argon in the dark for an additional 45 min. A sample of BF₃.OEt₂ (0.168 mL, 1.32 mmol) was added to the reaction mixture dropwise via syringe and the solution stirred under argon in the dark for 1 h, DDQ (0.68 g, 3.0 mmol) was added, and resulting mixture was stirred for an additional 1 h. A sample of triethylamine (13.2 mmol, 10 mol equiv versus BF₃.OEt₂) was added and the reaction mixture was stirred for 10 more min. The resulting crude reaction mixture was concentrated to dryness and chromatographed [silica, hexanes:CH₂Cl₂:CHCl₃ (30:1:1), 3 days slow elution, column was not pressurized] to afford purple solid (114 mg, 38%, yield is based on aldehyde 3). ¹H NMR (500 MHz, CDCl₃) δ/ppm: −2.75 (s, 2H), 1.24 (s, 9H), 1.56 (s, 9H), 1.91 (s, 6H), 7.03 (d, J=2.5 Hz, 1H), 7.4 (d, J=2.5 Hz, 1H), 7.92 (d, J=2.5 Hz, 1H), 8.05 (d, J=2.5 Hz, 1H), 8.79 (d, J=4.5 Hz, 2H), 8.91 (s, 4H), 8.97 (d, J=4.5 Hz, 2H); Anal. Calcd. for (M+H⁺), M=C₆₁H₃₈BrF₁₅N₄O: Calcd. 1207.2062. Found for HR(ESI)-MS: 1207.2108; LD-MS, 1205.42. λ_{max,abs}/nm (CH₂Cl₂)=416, 510, 544, 587, 639. λ_{max,em}(416 exc)/nm=642, 707.

### 5-(4-(5-Bromo-2,7-di-tert-butyl-9,9-dimethyl-xanthene))-10,15,20-tris(pentafluorophenyl)-porphyrin (HPX—Br, 2) (stepwise synthesis, Scheme 7)

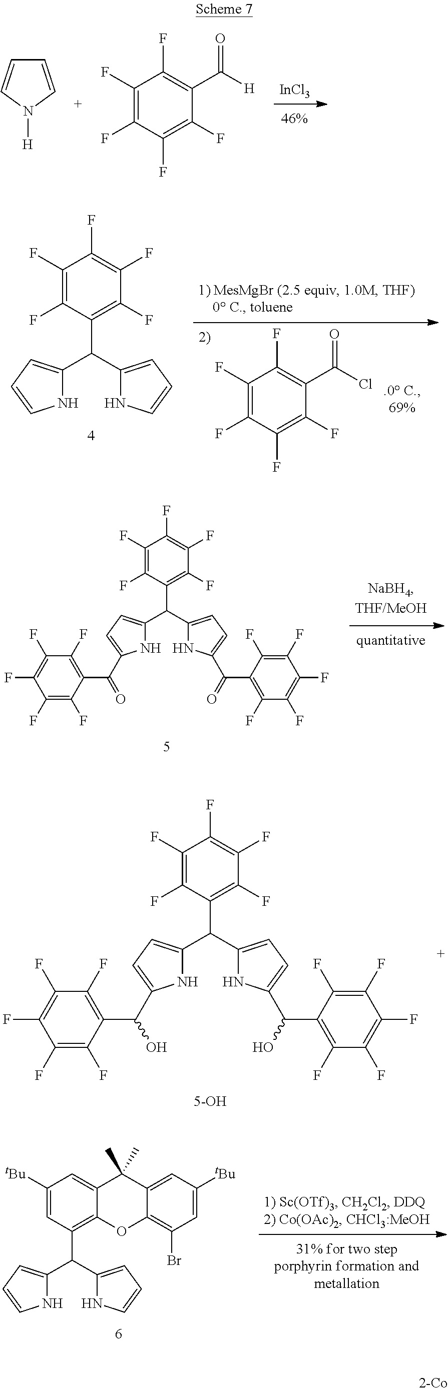

Following reported procedures, (Zaidi, S. H. H.; Fico, R. M., Jr.; Lindsey, J. S. Org. Proc. Res. Dev. 2006, 10, 118-134) a solution of 5 (0.350 g, 0.500 mmol) in dry THF/methanol (40 mL, 3:1) under argon at room temperature was treated with NaBH₄ (0.945 g, 12.5 mmol, 25 mol equiv versus 5) in small portions with rapid stirring. The progress of the reaction was monitored by silica thin layer chromatography (TLC) analysis using a hexanes/CH₂Cl₂ (1:1) mixture as the eluent. On the basis of TLC, the reaction was found to be complete in ˜30 min. The reaction mixture was poured into a solution of saturated aqueous NH₄Cl (100 mL) and ethyl acetate (100 mL). The organic phase was separated, washed with water and brine, dried over Na₂SO₄, and concentrated under reduced pressure at ambient temperature to yield the corresponding 1,9-diacyldipyrromethanedicarbinol (5-OH) as a yellow-orange foam-like solid. A sample of a dipyrromethane 6 (0.272 g, 0.500 mmol) was added into the flask contacting the 5-OH. The flask was fitted with a septum and it was then purged with argon for ˜10 min. Anhydrous CH₂Cl₂ (20 mL, 25 mM for each reactant) was added under a slow argon flow. The resulting reaction mixture was stirred for 1 min to produce a homogenous solution. Sc(OTf)₃ (0.003 g, 0.0650 mmol, 3.25 mM) was slowly added to this solution and the mixture was stirred for 30 min under argon. 2,3-Dichloro-5,6-dicyano-benzoquinone (DDQ) (0.340 g, 1.50 mmol) was added. After stirring at room temperature for 1 h, the flask was charged with triethylamine (0.175 mL, 1.28 mmol). The reaction was stirred 10 min and concentrated to dryness. The resulting crude product was dissolved in CH₂Cl₂ (100 mL), washed with water and brine, dried over Na₂SO₄ and concentrated to dryness. The crude product was subject to silica chromatography [hexanes:CH₂Cl₂ (4:1)] to afford purple solid (192 mg, 32%). The characterization data is consistent with the batch obtained by statistical synthesis.

5-(4-(5-Bromo-2,7-di-tert-butyl-9,9-dimethylxanthene))-10,15,20-tris(pentafluorophenyl)-porphyrinatocobalt (2-Co). By modifying published procedures, a microwave glass tube (10 mL) containing a magnetic stir bar was charged with 7 mL of CHCl₃:MeOH (3:1) and 2 (0.0650 g, 0.0540 mmol). The solution was stirred at room temperature for 10 min to obtain a homogenous mixture. A sample of Co(OAc)₂ was added (0.0500 g, 0.270 mmol, 10 mol equiv versus 2). The resulting mixture was stirred at room temperature for 5 min. The reaction vessel was sealed with a septum and subjected to microwave irradiation at 65° C. The protocol was as follows: (1) heat the reaction vessel from room temperature to 65° C., (2) hold at 65° C. and irradiate for 20 min (temperature overshoots of 67-70° C. were permitted; temperature was re-established at 65° C. by using open flow valve option), (3) allow the reaction mixture to cool to room temperature, (4) check the reaction mixture by silica TLC analysis, (5) repeat steps 1-4 until all of the free base 2 starting material was consumed (9-12 h). Upon complete reaction, triethylamine (10 mol equiv to metal salt) was added to the solution, which was washed with water and brine, dried over Na₂SO₄, and concentrated to dryness. The resulting crude product was chromatographed [silica, hexanes:CH₂Cl₂: CHCl₃ (8:1:1)] to afford dark red-orange solid (65 mg, 96%). ¹H NMR (500 MHz, CDCl₃) δ/ppm: 0.99 (s, 9H), 2.47 (s, 9H), 2.94 (s, 6H), 5.77 (s, 1H), 7.83 (s, 1H), 9.58 (s, 1H), 12.2-12.4 (br.s, 1H), 14.54-15.58 (br. s. 8H); Anal. Calcd. for (M⁺), M=C₆₁H₃₆BrCOF₁₅N₄O: Calcd. 1263.1159. Found for HR(ESI)-MS: 1263.1144; LD-MS. 1264.03. Anal. Calcd. for C₆₁H₃₆BrCoF₁₅N₄O: C, 57.93; H, 2.87; N, 4.43. Found: C, 58.17; H, 3.06; N, 4.26. λ_{max,abs}/nm (CH₂Cl₂)=407, 526.

5-(4-(5-Bromo-2,7-di-tert-butyl-9,9-dimethyl-xanthene))-10,15,20-tris(pentafluorophenyl)-porphyrintozinc (ZnHPX—Br, 2-Zn). By modifying published procedures a sample of 2 (0.0650 g, 0.0540 mmol) in CHCl₃:MeOH (15 mL 4:1) was treated with Zn(OAc)₂.2H₂O (0.295 g, 1.35 mmol, 25 mol equiv vs 2) at room temperature. The reaction mixture was stirred overnight. The reaction mixture was washed with water, brine, dried with Na₂SO₄ and concentrated to dryness. The resulting crude product chromatographed [silica, hexanes: CH₂Cl₂ (1:3)] afforded a purple solid. (67 mg, 97%). ¹H NMR (500 MHz, CDCl₃) δ/ppm: 1.21 (s, 9H), 1.55 (s, 9H), 1.89 (s, 6H), 6.98 (d, J=2.5 Hz, 1H), 7.38 (d, J=2.5 Hz, 1H), 7.89 (d, J=2.5 Hz, 1H), 8.08 (d, J=2.5 Hz, 1H), 8.87 (d, J=4.5 Hz, 2H), 8.98 (s, 4H), 9.06 (d, J=4.5 Hz, 2H); Anal. Calcd. for (M+H⁺), M=C₆₁H₃₆BrF₁₅N₄OZn: Cald. 1271.1195. Found for HR(ESI)-MS: 1271.1180. λ_{max,abs}/nm (CH₂Cl₂)=417, 546. λ_{max,em}(417 exc)/nm=589, 642.

Electrochemistry. Electrochemical experiments were performed with a BASi CV50W in a glove box. Cyclic voltammetry (CV) experiments were performed using a glassy carbon working electrode (0.07 cm²), a platinum wire auxiliary electrode and a Ag/AgNO₃ (0.1 M) reference electrode in 0.1 M NBu₄PF₆ acetonitrile solution at room temperature. NBu₄PF₆ was dried at 120° C. and acetonitrile was purified by passing them under an argon forcing pressure through columns of neutral alumina. Tosic acid monohydrate (Aldrich) and benzoic acid (Aldrich) were used as received. A polished electrode was used for each CV. The potentials were referenced to ferrocene/ferrocenium couple by recording the CV of the complexes in the presence of a small amount of ferrocene.

Bulk electrolysis was performed using a glassy carbon rod (7 mm×5 cm) working electrode and a platinum mesh auxiliary electrode in a gas-tight electrochemical cell. The amount of H₂ gas produced in the headspace was analyzed by an Agilent 7890A GC. The potentials for the electrolyses (−2.05 V for 1-Co and −2.20 V vs Fc/Fc⁺ for 2-Co) were referenced with Co^{II/I} redox couples in the CV obtained before adding acid solution.

Cyclic voltammograms for 1-Co and 2-Co in the presence of benzoic and tosic acids were obtained. The independence of the first oxidation event of 2-Co in acid concentration is evident. Cyclic voltammograms for benzoic and tosic acids were obtained. The CV of 2-Zn was also obtained.

CV of 0.5 mM 1-Co in the presence of 0 (i), 7.0 (ii) and 14.6 mM (iii) benzoic acid; CV of 0.5 mM 2-Co in the presence of 0 (i), 7.5 (ii) and 15 mM (iii) benzoic acid; CV of 0.8 mM 1-Co in the presence of 0 (i), 5.0 (ii), 10 (iii), 20 (iv) mM tosic acid; CV of 0.8 mM 2-Co in the presence of 0 (i), 5.0 (ii), 10 (iii), 20 (iv) mM tosic acid; CV of 7.5 (i) and 15.0 mM (ii) benzoic acid; CV of 5 (i), 10 (ii) and 20 mM (iii) tosic acid; CV of 0.5 mM 2-Co in the presence of 0 (i), 7.5 (ii) and 15.0 mM (iii) benzoic acid; Cyclic voltammogram of 2-Zn. Scan rate, 25 mV/s; were all obtained.

The absorption and emission spectrum of 2 in CH₂Cl₂ at room temperature showed peaks at 416 (max), 510, 587, and 639 nm (absorption), and 544, 642, and 707 (max) nm (emission), respectively.

The absorption spectrum of 2-Co in CH₂Cl₂ at room temperature showed peaks at 407 (max) and 526 nm.

The absorption and emission spectrum of 2-Zn in CH₂Cl₂ at room temperature showed peaks at 417 (max) and 546 nm (absorption), and 589 and 642 (max) nm (emission), respectively.

### Example 5

Cobalt hangman porphyrins can catalyze the hydrogen evolution reaction (HER). The hangman group was observed to facilitate HER by mediating a proton-coupled electron transfer (PCET) reaction. The details of the PCET pathway were determined by comparing rate constants associated with the ET and PT processes of the hangman system to those of the corresponding values measured for porphyrins that lack an internal proton relay. A rapid intramolecular proton transfer from the carboxylic acid hanging group to the reduced cobalt centre of 8.5×10⁶ s⁻¹ provided a facile pathway for the formation of Co(II)H, which led to H₂ generation.

The hydrogen evolution reaction (HER) is requisite for solar-to-fuels production. To avoid energy-wasting reaction barriers, it is desirable to have the electron and proton coupled throughout the reaction profile of the HER transformation. To enable this coupling, proton-accepting or proton-donating groups in the second coordination sphere of redox centers has been shown to be particularly useful for enhanced catalytic efficiency of the HER as well as other energy conversion processes including the oxygen reduction reaction and its reverse, the oxygen evolution reaction. The hangman motif may be especially attractive for promoting proton-coupled electron transfer (PCET) conversions of small molecule substrates. The hangman moiety positions an acid/base group above a redox-active metal platform. The hanging group serves as a proton shuttle to deliver protons to and/or accept protons from substrates bound to the metal centre and in doing so, hangman constructs enable control over the nature of the acid/base environment directly adjacent to the redox site. The hangman motif has been particularly attractive for the HER. Cobalt hangman porphyrin 11 (Scheme 8) serves as a HER electrocatalyst as evidenced by at least the enhanced HER efficiency and a shift of the catalytic wave to lower overpotentials compared to analogous non-hangman systems. For this conversion, the Co(II)H species was an important intermediate for promoting HER turnover. This example describes the kinetic studies that investigate the details of the PCET pathway involved in the formation of the Co(II)H species and describe the kinetic parameters that govern the formation of this intermediate.

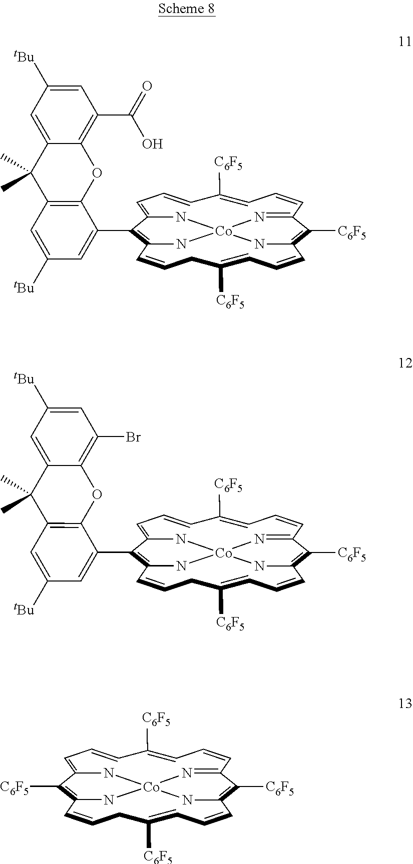

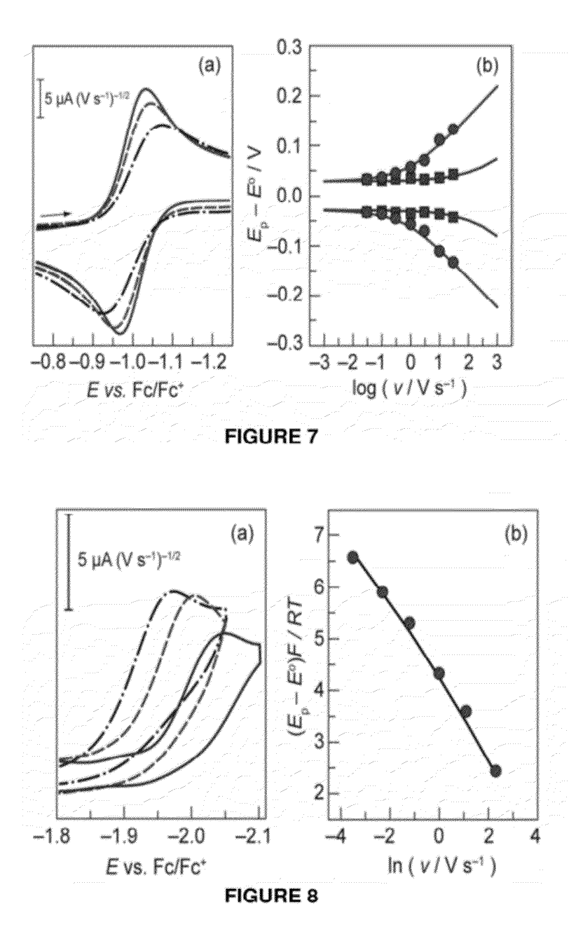

Rate constants associated with the electron transfer (ET) and proton transfer (PT) processes of hangman compound II, were determined and compared with the corresponding values measured for 12 and 13 (Scheme 8), which lack an internal proton relay. Porphyrins 11-13 were synthesized in short synthesis times and in good yields (e.g., as described herein). FIG. 7a shows the voltammetric waves of the formal Co^{2+/+} couple of 13 acquired from cyclic voltammetry (CV) over a range of scan rates. For simplicity, the formal metal oxidation state is used in this example, but those of ordinary skill in the art will understand that the metal orbitals are mixed with the porphyrin ring and hence the formal reduction of the metal may involve delocalization of the reducing equivalent onto the porphyrin ring. As the scan rate is increased, the onset of electrochemical irreversibility is generally observed as the peak separation between anodic and cathodic waves increases. Under these conditions, the chemical reaction may be influenced by the kinetics of the electron transfer process, as opposed to operating under the conditions of diffusion control (indicated by a 60 mV separation at 25° C.). The “trumpet plot” for the Co^{2+/+} couple (e.g., FIG. 7b) of 13 shows the difference between the anodic or cathodic peak potential and midpoint potential (Eₚ−E^{o}) as a function of the logarith of scan rate (log ν). Simulation of CVs to replicate the shape of the trumpet plot furnished a value of 0.011 cm s⁻¹ for standard heterogeneous electron transfer rate constant, kₛ, of the Co^{2+/+} couple. The trumpet plot for the Co^{+/0} couple of 13 is also shown in FIG. 7b. The kₛ extracted from the trumpet plot for the Co^{+/0} couple of 13 was at least an order of magnitude higher than that of the Co^{2+/+} couple, on the order of 0.2 cm s⁻¹. The kₛ of 0.012 cm s⁻¹ for the Co^{2+/+} couple of 11 was the same as that of 13. The presence of the carboxylic acid hanging group in 11 can result in irreversibility of the Co^{+/0} redox wave owing to an irreversible reaction between the reduced metal center and the internal acid group. The Co(0) was observed to protonate to produce a Co(II)H species, which in the presence of excess acid reacted in an ensuing step to produce H₂. The presence of the hanging group resulted in a lower onset overpotential for H₂ production by ˜200 mV compared to non-hangman systems such as 12.

In FIG. 7: (a) Overlay of representative normalized (iₙₒᵣₘ=i/ν^{1/2}) (i is current, ν is scan rate) CVs taken of a 0.3 mM solution of cobalt porphyrin 13 in acetonitrile with 0.03 ( [symbol] ), 0.3 ( [symbol] ), and 3 ( [symbol] ) V s⁻¹ scan rates, using a glassy carbon electrode. (b) Difference between anodic or cathodic peak potential and midpoint potential (Eₚ−E^{o}) vs. log of scan rate (E^{o}=−1.00 V for the Co^{2+/+} couple (), and −1.98 V for the Co^{+/0} couple (▪)). Simulated curves are plotted for kₛ=0.011 cm s⁻¹ ( [symbol] ) and kₛ=0.2 cm s⁻¹ ( [symbol] ). The diffusion coefficient (D) of 13 was determined to be 8×10⁻⁶ cm² s⁻¹ from the peak current, i, in the reversible limit: i=0.446FAC^{o}D^{1/2}(Fv/RT)^{1/2} (F is the faraday constant, A is the area of the electrode and C^{o} is the bulk porphyrin concentration).

The similarity in Co^{2+/+} midpoint potential between 11 and 12 may suggests that the position of the reversible Co^{+/0} wave of 12 (E^{o} at −2.14 V vs. Fc/Fc⁺) is a good estimate of the Co^{-/0} midpoint potential of 11 as well. The position of the irreversible Co^{+/0} peak to more positive potentials than the expected reversible potential may be indicative of what is classically considered an EC mechanism, where a heterogeneous electron transfer reaction is followed by a homogeneous chemical reaction. In hangman-promoted HER, the “C” step may involve an intramolecular proton transfer from the hanging group to the metal center to furnish the hydridic Co(II)H species. This EC mechanism for a hangman platform is generally as follows,

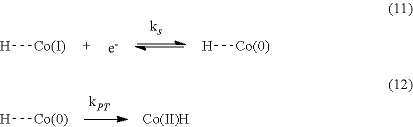

where H—Co indicates the presence of a proton in the secondary coordination sphere of the cobalt centre. Such an EC mechanism within a PCET context is ETPT. In this case, the peak potential of a CV (Eₚ) may be independent of the bulk concentration of the complex. In addition, when electron transfer is reversible and fast enough so as not to interfere kinetically in the electrochemical response, Eₚ may be expressed as a function of the reversible potential of the ET step (E^{o}), the proton transfer rate constant (k_{PT}), and the scan rate (ν) as follows,

$${E}_{p} = {E}^{o} - 0.78 \frac{RT}{F} = \frac{RT}{2 F} ln ( \frac{RT}{F} \frac{{k}_{PT}}{v} ) \qquad ( 13 )$$

The peak potential of the Co^{+/0} couple of 11 was independent of the concentration of 11 over a range of scan rates. To determine the rate constant associated with the intramolecular proton transfer between the hanging group and the metal center, CVs were acquired by varying the scan rate between 0.03 and 30 V s⁻¹ (FIG. 8a) at a fixed metalloporphyrin concentration of 0.2 mM. Eq. 13 predicted a linear correlation between the dimensionless parameter (Eₚ−E^{o})F/RT and ln ν with a slope of −0.5. The experimental data, however, displayed a distinct curvature that became more pronounced at higher scan rates (FIG. 8b). These observations are consistent with kinetic competition between the heterogeneous ET step (Eq. 11) and the irreversible homogeneous PT (Eq. 12). As the scan rate was increased, effects attendant to passage into a regime where the ET (along with diffusion) limits the electrochemical response were observed. Although this complication precluded the straightforward determination of k_{PT} from Eq. 13, the presence of this mixed control permitted determination of the kinetic parameters governing ET and PT steps from a simulation of the dependence of peak potential on scan rate. The peak potential of the slow scan (30 mV s⁻¹) voltammogram was substituted into Eq. 13 to obtain an initial estimate of the intramolecular PT rate constant of 3×10⁶ s⁻¹. In addition, the k₅=0.2 cm s⁻¹ associated with the Co^{+/0} couple of 13 (FIG. 7b) was used as an initial approximation for that of the hangman system. These parameters were optimized by CV simulation iteratively until the simulated peak potentials over the range of scan rates agreed with the experimental data, resulting in the fit displayed in FIG. 8b. The results of this fit yielded a heterogeneous ET rate constant of 0.24 cm s⁻¹ for Eq. 11 and k_{PT}=8.5×10⁶ s⁻¹ for the formation of the Co(II)H species via Eq. 12.

In FIG. 8: (a) Overlay of representative CVs taken of a 0.2 mM solution of 11 in acetonitrile with 0.03 ( [symbol] ), 0.3 ( [symbol] ), and 3 ( [symbol] ) V s scan rates, using a glassy carbon electrode. (b) Plot of (Eₚ−E^{o})F/RT vs. In of scan rate (E^{o}=−2.14 V, taken from system 12). Experimental data (), simulated curve ( [symbol] ) plotted for kₛ=0.24 cm s⁻¹ and k_{PT}=8.5×10⁶ s⁻¹.

In order to compare the kinetics of the intramolecular PT for 11, to an intermolecular PT from an external acid source, CVs of 13 were recorded in the presence of varying amounts of benzoic acid (FIG. 8a). The Co^{+/0} redox wave became irreversible in the presence of excess benzoic acid and a catalytic wave was observed in the CV. In this case, catalytic H₂ production was observed upon reduction to Co(0), and thus, a second protonation step must be taken into consideration. Kinetic resolution of the system requires knowledge of which of the two following protonation steps is rate-limiting in the overall catalytic reaction:

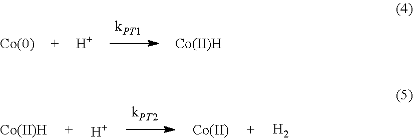

There are three parameters that govern the rate of H₂ evolution catalysis, and therefore the magnitude of the peak catalytic current (i_{cat}):
(i) the ratio of substrate (acid) to catalyst (porphyrin) concentration, i.e. the excess factor (γ):

$$γ = \frac{{[ PhCOOH ]}_{bulk}}{{[ 3 ]}_{bulk}} \qquad ( 16 )$$

(ii) the dimensionless parameter λ_{PT1} which defines the kinetics of Eq. 14:

$${λ}_{PT 1} = \frac{RT}{F} \frac{{[ 3 ]}_{bulk} {k}_{PT 1}}{v} \qquad ( 17 )$$

(iii) the competition between Eqs. 4 and 5, given by ρ where:

$${λ}_{PT 1} = \frac{RT}{F} \frac{{[ 3 ]}_{bulk} {k}_{PT 1}}{v} \qquad ( 17 )$$

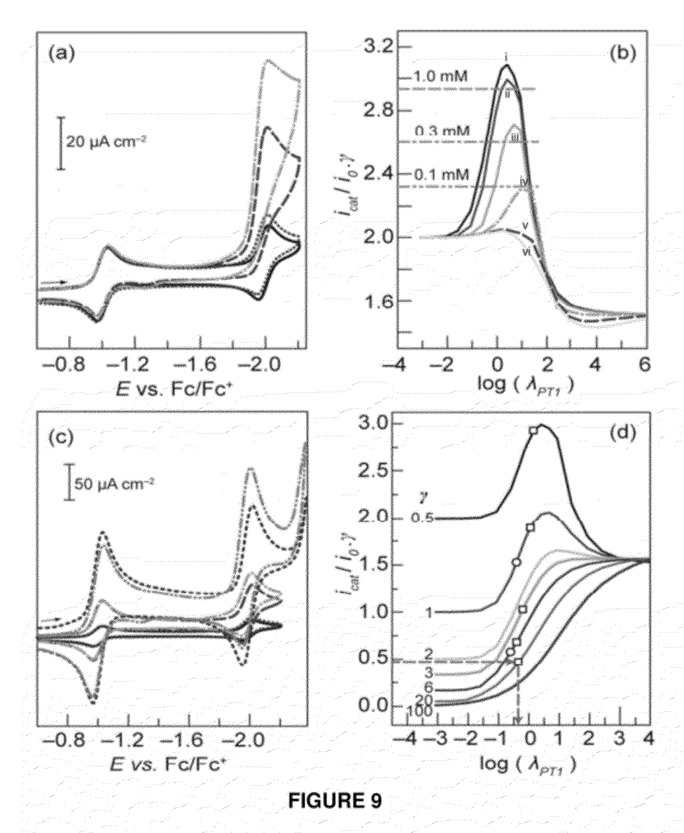

In general, Eq. 14 is rate-limiting for ρ>10 and Eq. 15 is rate-limiting for ρ<0.1. Because the waves for catalysis and for the catalyst in the absence of acid occur at similar potentials, the peak in the CV may be due to both substrate and catalyst consumption. The confluence of these two processes precludes an analytical solution to the problem at hand, and generally requires the use of CV simulation to generate working curves that relate measurable quantities (e.g., peak current values) to kinetic parameters. In order to determine which step is rate-limiting, working curves were generated as follows. A CV was simulated for the case of a catalyst at a specified concentration in the absence of substrate (PhCOOH). The peak current associated with the Co^{+/0} couple was recorded as i₀. Next, a CV was simulated for the same catalyst concentration, but with substrate added at an excess factor, γ, of 0.5 for a reaction scheme in which k_{PT2} is 100 times a specified value of k_{PT1} (ρ=100). The peak current associated with this voltammogram was recorded as i_{cat}. The normalized current value i_{cat}/i₀γ was calculated. Varying k_{PT1} (and thus log μ_{PT1}) while maintaining γ=0.5 and ρ=100, and determining the corresponding i_{cat} (and therefore i_{cat}/i₀γ) values, furnished the curve (i) in FIG. 9b. This was repeated for ρ=10, 1, 0.1, 0.01, and 0.001 to generate the remaining plots in FIG. 9b. The maxima in these working curves define the maximum normalized current values attainable, for a specific ρ.

To determine the value of ρ that was operative during H₂ production mediated by 13, and therefore the identity of the rate-limiting step, CVs of solutions containing 0.1, 0.3, and 1 mM 13 were recorded (FIG. 9c), and the Co^{+/0} peak currents (i₀) were determined in each case. CVs were also acquired for solutions with identical concentrations of 13, but containing 0.5 equivalents of benzoic acid (i.e. γ=0.5) (FIG. 9c), permitting measurement of the corresponding catalytic peak currents (i_{cat}). The resultant normalized current ratios (i_{cat}/i₀γ), that were generated (designated by horizontal lines in FIG. 9c) are consistent with Eq. 14 as the rate-limiting step (ρ≧10), since in the case of 1 mM catalyst concentration, i_{cat}/i₀γ=2.9 (FIG. 9b). In contrast, the maximum value for the cases where ρ≦1 is 2.7. Notably, since Eq. 14 appears to be limiting in this embodiment, the precise value of ρ was not be determined; the working curves associated with ρ=100 and ρ=10 are almost identical, and the discrepancy between ρ=1000 and ρ=100 can be shown to be even less. For that matter, whereas k_{PT1} may be determined, k_{PT2} generally cannot.

To estimate the value of k_{PT1}, working curves of i_{cat}/i₀γ vs. log λ_{PT1} based on the i_{cat} and i₀ values extracted from simulated CVs were generated. In this case, ρ=10 was fixed for all simulations (since this furnishes the upper limit for k_{PT1} compared to all other ρ>10) and varied λ_{PT1} for different values of the excess factor, γ (FIG. 9c). Therefore, one value of γ is associated with each curve in FIG. 9d. Experimental i_{cat} and i₀ values were then obtained from CVs of a 1.0 mM solution of 13 with increasing concentrations of benzoic acid between 0 (affording i₀) and 20 mM. Normalized current ratios (i_{cat}/i₀γ) from these CVs give five distinct points on the vertical axis of the i_{cat}/i₀γ vs. log λ_{PT1} plots in FIG. 9d. Each one of these values corresponded to a point on the specific working curve associated with the benzoic acid concentration (γ value) of that CV. For each point on the working curves, a log λ_{PT1} value may be determined (arrows, FIG. 9d). Each dimensionless parameter, λ_{PT1}, thus obtained permitted the calculation of a discrete value of k_{PT1} (Eq. 17). The calculated rate constants ranged between ˜400 to 1600 M⁻¹ s⁻¹. The intermolecular (2^{nd} order) PT rate constant between benzoic acid and 13 in its reduced Co(0) state was determined to be on the order of 1000 M⁻¹ s⁻¹. Previous experimental CV data (i₀ and i_{cat} values) from the titration of 12 with benzoic acid, was evaluated in an similar manner to yield a similar intermolecular PT rate constant of −2500 M⁻¹ s⁻¹ for the reaction between the Co(0) center in 12 and benzoic acid. In comparison to these intermolecular rate constants, the measured intramolecular PT rate constant of 8.5×10⁶ s⁻¹ for 11 may indicate that the presence of a pendant proton relay proximate to the metal center gives rise to a rate enhancement that is equivalent to an effective benzoic acid concentration >3000 M.

In FIG. 9: (a) CVs of a 0.1 mM solution of 13 in the absence of benzoic acid ( [symbol] ) and in the presence of 0.05 ( [symbol] ) 0.3 ( [symbol] ), and 0.6 ( [symbol] ) mM benzoic acid. Scan rate, 30 mV s⁻¹; 0.2 M TBAPF₆ in acetonitrile. Glassy carbon working electrode, Ag/AgNO₃ reference electrode, and Pt wire counter electrode. (b) Working curves generated by simulating CVs with γ=0.5 and ρ=100 (i), 10 (ii), 1 (iii), 0.1 (iv), 0.01 (v), and 0.001 (vi). Horizontal lines indicate normalized current values (i_{cat}/i₀γ) obtained at the designated porphyrin concentrations, with half an equivalent of benzoic acid. (c) CVs of 0.1 ( [symbol] ), 0.3 ( [symbol] ), and 1 mM ( - - - ) solutions of 13 in the absence of benzoic acid and in the presence of 0.05 ( [symbol] ) 0.15 ( [symbol] ), and 0.5 ( [symbol] ) mM benzoic acid, respectively. Scan rate, 30 mV s⁻¹; 0.2 M TBAPF₆ in acetonitrile. Glassy carbon working electrode, Ag/AgNO₃ reference electrode, and Pt wire counter electrode. (d) Working curves generated by simulating CVs with ρ=10. Experimental data points for 13 (□) and 12 (∘) acquired at scan rates of 30 and 100 mV s⁻¹ respectively.

The PCET kinetics attendant to the HER activity of cobalt hangman porphyrins were examined. A comparison between the PCET kinetics of 11 and non-hangman systems 12 and 13 aids in establishing the hangman effect. The rate constant for transfer of a proton from the carboxylic acid hanging group to the reduced cobalt centre was 8.5×10⁶ s⁻¹. The rapid intramolecular proton transfer appears to provide a facile pathway for the formation of Co(II)H, which reacts with protons to lead to H₂ generation. The presence of the hanging carboxylic acid group in 11 is important in the enhanced H₂ electrocatalysis and is evidence for the “hangman effect” in promoting HER.

### Example 6

This example provides supporting information for Example 3.

Materials: Catalysts 11 (Co—HPXCOOH), 12 (Co—HPXBr), and 13 (Co(C₆F₅)₄) were prepared following published procedures (e.g., see C. H. Lee, D. K. Dogutan and D. G. Nocera, J. Am. Chem. Soc., 2011, 133, 8775-8777; D. K. Dogutan, D. K. Bediako, T. S. Teets, M. Schwalbe and D. G. Nocera, Org. Lett., 2010, 12, 1036-1039; R. McGuire Jr., D. K. Dogutan, T. S. Teets, J. Suntivich, Y. Shao-Horn and D. G. Nocera, Chem. Sci., 2010, 1, 411-414). Benzoic acid (≧99.5%) and tetrabutylammonium hexafluoro-phosphate (TBAPF₆, ≧99.0%) were purchased from Aldrich and used as received.

Electrochemical studies: Electrochemical measurements were performed on a CH Instruments (Austin, Tex.) 760D Electrochemical Workstation using CHI Version 10.03 software. Cyclic voltammetry (CV) experiments were conducted in a nitrogen-filled glovebox at 295 K using a CH Instruments glassy carbon button working electrode (area=0.071 cm²), BASi Ag/AgNO₃ reference electrode, and Pt mesh counter electrode in 0.2 M TBAPF₆ acetonitrile solutions 2 or 4 mL total volume. Acetonitrile was previously dried by passage through an alumina column under argon. All CVs were recorded with compensation for solution resistance, and were referenced to the ferrocene/ferrocenium (Fc/Fc⁺) couple by recording the CVs of the complexes in the presence of a small amount of ferrocene. Appropriate background scans were subtracted from all CVs. Solutions were stirred between acquisition of individual CVs and the working electrode was polished before each measurement.

The concentration dependence of the Co^{+/0} peak potential of 11 was determined by preparing a 1 mM solution of 11 in 0.2 M TBAPF₆ and successively diluting this solution with electrolyte to afford solutions of 0.75, 0.5, and 0.25 mM 11. A CV of each solution was recorded at 0.03, 0.3, and 3 V s⁻¹.

In the case of the titration experiment shown in FIG. 9a, to a 2 mL of 0.1 mM 13 solution in 0.2 M TBAPF₆ was added, 2, 10, and 12 μL of a 50 mM benzoic acid solution in 0.2 M TBAPF₆ to afford solutions comprising 0.05, 0.3, and 0.6 mM benzoic acid. The change in the total volume of the sample, and thus, in the concentration of 3, was negligible.

In the case of the titration experiments shown in FIG. 9c, for the CVs that correspond to 0.1 mM 13 ( [symbol] and [symbol] lines), to a 2 mL of 0.1 mM 3 solution in 0.2 M TBAPF₆, 2 μL of a 50 mM benzoic acid solution in 0.2 M TBAPF₆ was added to afford a solution comprising 0.05 mM benzoic acid. The change in the total volume of the sample, and thus, in the 13 concentration, was negligible. The CV of the solution was recorded at the beginning for the acid free sample ( [symbol] line in FIG. 9c) to get the i₀ value, and after the benzoic acid addition ( [symbol] line in FIG. 9c) to get the i_{cat} value. For the CVs that correspond to 0.3 mM 13 ( [symbol] and [symbol] lines in FIG. 9c), to a 2 mL of 0.3 mM 13 solution in 0.2 M TBAPF₆, 2 μL of a 150 mM benzoic acid solution in 0.2 M TBAPF₆ was added to afford a solution comprising 0.15 mM benzoic acid. The change in the total volume of the sample, and thus, in the concentration of 13, was negligible. The CV of the solution was recorded at the beginning for the acid free sample ( [symbol] line in FIG. 9c) to get the i₀ value, and after the benzoic acid addition ( [symbol] line in FIG. 9c) to get the i_{cat} value.

For the CVs that correspond to 1 mM 13 ( - - - and [symbol] lines in FIG. 9c), stock solutions of 2 mM 13 in 0.2 M TBAPF₆ and 100 mM benzoic acid in 0.2 M TBAPF₆ were initially prepared. 2 mL of the 2 mM 13 solution were diluted with electrolyte until final volume 4 mL to give a 1 mM 13 sample. The CV of this acid free solution was recorded ( - - - line in FIG. 9c) to obtain the i₀ value. Then, 2 mL of the 2 mM 13 solution, 20 μL of the 100 mM benzoic acid solution and 1980 μL electrolyte (0.2 M TBAPF₆) were mixed together to give a 4 mL solution comprising 1 mM 13 and 0.5 mM benzoic acid. The CV of the latter was recorded ( [symbol] line in FIG. 9c) to get the i_{cat} value. The two stock solutions were also used for the titration experiment described below.

For the titration experiment of a 1 mM 13 solution with benzoic acid, stock solutions of 2 mM 13 in 0.2 M TBAPF₆ and 100 mM benzoic acid in 0.2 M TBAPF₆ were initially prepared. 2 mL of the 2 mM 13 solution were diluted with electrolyte until final volume 4 mL to give a 1 mM 13 sample. The CV of this acid free solution was recorded to obtain the i₀ value. Appropriate volumes from the two stock solutions were mixed with electrolyte until final volume 4 mL to give the corresponding 1 mM 13 solutions with 0.5, 1, and 3 mM benzoic acid. To the latter (i.e. 3 mM) solution, 1.5 and 6.8 mg of benzoic acid was successively added to afford solutions comprising 6 and 20 mM acid concentrations. The CVs of all the above acidic solutions were recorded to get the corresponding i_{cat} values.

CV simulation and generation of working curve: All simulated CVs were calculated using the DigiElchsoftware package. Diffusion coefficients of compounds were determined straightforwardly from the peak currents of reversible waves, and these values were used in the applicable simulations. Symmetry factors (α values) were set as 0.5 for all ET steps.

The working curves shown in FIG. 9b were generated as follows: using the experimentally determined heterogeneous rate constants a CV was simulated for the case of a catalyst at a specified concentration in the absence of substrate (PhCOOH). The peak current associated with the Co^{+/0} couple was recorded as i₀. Next, a CV was simulated for the same catalyst concentration, but with substrate added at an excess factor, γ, of 0.5 for a reaction scheme in which k_{PT2} is 100 times a specified value of k_{PT1} (ρ=100). The peak current associated with this voltammogram was recorded as i_{cat}. The normalized current value i_{cat}/i₀γ was calculated. Varying k_{PT1} (and thus, according to Eq. 17, log λ_{PT1}), and determining the corresponding i_{cat} (and therefore i_{cat}/i₀γ) values, furnished the curve (i) in FIG. 9b. Although this may also be achieved by modulating other variables (i.e. T, ν, or [13]_{bulk}) as described by Eq. 17, alteration of any other variable would also require simulation of a new CV in the absence of substrate to acquire a new i₀. As k_{PT1} was altered, k_{PT2} was set to the appropriate value fixed by the desired ratio of k_{PT2} to k_{PT1}, ρ (Eq. 18). This was repeated for ρ=10, 1, 0.1, 0.01, and 0.001 to generate the remaining plots in FIG. 9b.

The working curves shown in FIG. 9d were generated as follows: using the experimentally determined heterogeneous rate constants a CV was simulated for the case of a catalyst at a specified concentration in the absence of substrate (PhCOOH). The peak current associated with the Co^{+/0} couple was recorded as i₀. Next, a CV was simulated for the same catalyst concentration, but with substrate added at an excess factor, γ, of 0.5 for a reaction scheme in which k_{PT2} is 10 times a specified value of k_{PT1} (ρ=10). The peak current associated with this voltammogram was recorded as i_{cat}. Thus, the normalized current value i_{cat}/i₀γ was calculated. Varying k_{PT1} (and thus, according to Eq. 17, log λ_{PT1}), and determining the corresponding i_{cat} (and therefore i_{cat}/i₀γ) values, furnished the 0.5 curve in FIG. 9d. As k_{PT1} was altered, k_{PT2} was set to the appropriate value fixed by the desired ratio of k_{PT2} to k_{PT1}, ρ=10 (Eq. 18). This was repeated for γ=1, 2, 3, 6, 20 and 100 to generate the remaining plots in FIG. 9d.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed:

1. A composition, having the formula:
X-Y;
wherein X comprises a macrocycle having 2-7 heteroatoms positioned such that at least some of the heteroatoms are able to coordinate a metal or a semi-metal within a central binding cavity of the macrocycle; and
Y is a pendent group, optionally substituted,
wherein at least one beta-position of the macrocycle is an electron-withdrawing group.

2. The composition of claim 1, wherein the electron-withdrawing group is selected from the group consisting of halide, NO₂, and CN.

3. A method, comprising:
forming a mixture of a metal complex comprising a metal atom and a composition having the formula:
X-Y;
wherein X comprising a macrocycle having 2-7 heteroatoms positioned such that at least some of the heteroatoms are able to coordinate a metal within a central binding cavity of the macrocycle, wherein at least one beta-position of the macrocycle is an electron-withdrawing group, optionally substituted; and
Y is a pendent group; and
exposing said mixture to microwave energy, thereby forming a compound comprising the macrocycle and the metal atom, wherein the metal atom is coordinated by at least some of the heteroatoms within the central binding cavity of the macrocycle.

4. The method of claim 3, wherein the electron-withdrawing group is selected from the group consisting of halide, NO₂ and CN.

5. A method of claim 3, wherein:
Y is substituted with at least one —Z—P_{g}, wherein Z is a hydrolyzable group and P_{g} is a protecting group; and
following exposure to microwave energy, Y is substituted with -Z-D_{g}, wherein D is a deprotected group or optionally absent.

6. The method of claim 3, further comprising reacting the compound following exposure to microwave energy having comprising the formula -Y-Z-D_{g}, to form a compound having the formula —Y—Z—H.

7. The method of claim 3, wherein Y is substituted with G, and wherein following exposing said mixture to microwave energy to form a compound comprising the macrocycle and the metal atom, wherein the metal atom is coordinated by at least some of the heteroatoms within the central binding cavity of the macrocycle and G.

8. A method of catalysis, comprising:
providing a composition having the formula:
X-Y;
wherein X comprises a macrocycle having 2-7 heteroatoms positioned such that at least some of the heteroatoms are able to coordinate a metal within a central binding cavity of the macrocycle, wherein at least one beta-position of the macrocycle is an electron-withdrawing group; and
Y is a pendent group, optionally substituted,
wherein at least one metal atom is coordinated by the macrocycle and/or the pendent group; and
exposing the composition to a reactant, wherein a product is formed from the reactant following application of a voltage to the composition.

9. The method of claim 8, wherein:
the catalysis comprises forming oxygen gas from water; and
wherein the exposing comprises exposing the composition to water, wherein oxygen gas is formed from water following application of a voltage to the composition.

10. The method of claim 8, wherein:
the catalysis comprises forming hydrogen gas from water; and
wherein the exposing comprises exposing the composition to water, acid, organic solvent, or combination thereof, wherein hydrogen gas is formed from the water, acid, organic solvent, or combination thereof following application of a voltage to the composition.

11. The method of claim 8, wherein:
the catalysis comprises reducing CO₂; and
wherein the exposing comprises exposing the composition to CO₂, wherein the CO₂ is reduced following application of a voltage to the composition.

12. The method of claim 8, wherein the electron-withdrawing group is selected from the group consisting of halide, NO₂, and CN.

13. The composition of claim 1, wherein the macrocycle comprises a compound having the structure: 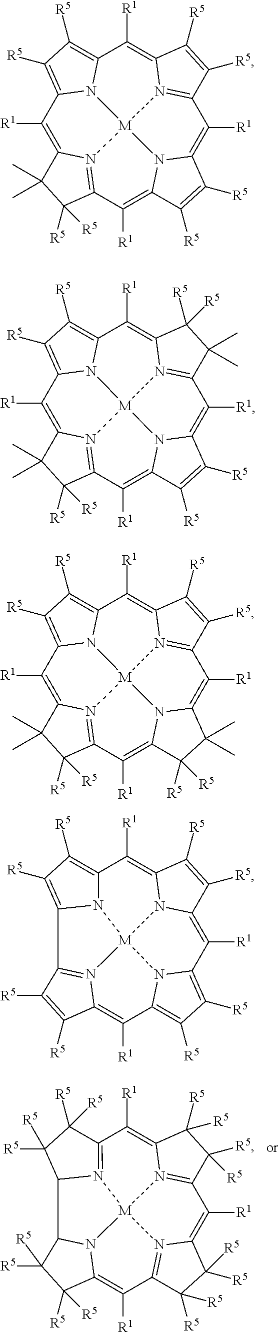 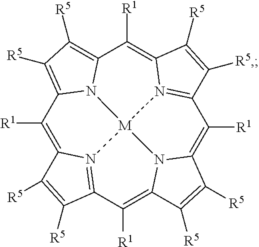
wherein each R¹ can be the same or different and is selected from the group consisting of hydrogen, alkyl, aryl, heteroalkyl, heteroaryl, alkenyl, alkynyl, cycloalkyl, cycloalkylalkyl, cycloalkylalkenyl, cycloalkylalkynyl, acyl, carboxylic acid, acylamino, alkylthio, amino, alkylamino, arylalkylamino, alkoxy, arylalkyl, or alkylaryl, each optionally substituted provided at least one R¹ is a group comprising of the formula —Y, optionally substituted;
wherein each R⁵ can be the same or different and is hydrogen, halide, CN, CO₂, alkyl, aryl, heteroalkyl, heteroaryl, alkenyl, alkynyl, cycloalkyl, cycloalkylalkyl, cycloalkylalkenyl, cycloalkylalkynyl, acyl, carboxylic acid, carboxylate, OH, acylamino, alkylthio, amino, alkylamino, arylalkylamino, or alkoxy, each optionally substituted, provided at least one R⁵ is an electron-withdrawing group; and
M is a metal atom, a semi-metal atom, or at least one hydrogen.

14. The composition of claim 1, wherein Y is substituted by -G, or —Z—P_{g}, or -Z-D, or —Z—H.

15. The composition of claim 1, wherein Y is substituted by —COOR², wherein R² is hydrogen, alkyl, aryl, heteroalkyl, heteroaryl, or a protecting group, each optionally substituted.

16. The composition of claim 13, wherein each R⁵ is an electron-withdrawing group.

17. The composition of claim 1, wherein the macrocycle is selected from the group consisting of a porphycene, a [18]porphyrin(2.1.0.1), an N-confused porphyrin, a sapphyrin, a heterosapphyrin, a rubyrin, an orangarin, a cycle[8]pyrrole, a rosarin, a turcasarin, a texaphyrin, a cryptan, a calixphyrin, or a catenane, each optionally substituted.

18. The composition of claim 14, wherein —Z—P_{g} is selected from the group consisting of —COOP_{g}, —PO(OR)(OP_{g}), —B(OR)(OP_{g}), —CO(NR)(NP_{g}), —NRP_{B}, —C(NR₂)(NRP_{g}), and —OP_{g}, wherein R is a suitable organic substituent and P_{g} is a protecting group.

19. The composition of claim 14, wherein -Z-D_{g} is selected from the group consisting of —COOD_{g}, —PO(OR)(OD_{g}), —B(OR)(OD_{g}), —CO(NR)(ND_{g}), —NRD_{g}, —C(NR₂)(NR)D_{g}), —OD_{g}, wherein R is a suitable organic substituent and D_{g} is a deprotected group or optionally absent.

20. The composition of claim 13, wherein M is a metal atom or a semi-metal atom.

21. The composition of claim 1, wherein Y comprises xanthene, dibenzofuran, biphenylene, or anthracene.

22. The composition of claim 1, wherein the composition comprises at least one substituent which aids in increasing the water solubility of the composition.
